(19) United States
(12) Patent Application Publication
(10) Pub. No.: US 2009/0188211 A1
(43) Pub. Date: Jul. 30, 2009

(54) Bag wrinkle remover, leak detection systems, and electromagnetic agitation for liquid containment systems

(75) Inventors: Parrish M. Galliher, Littleton, MA (US); Michael Fisher, Ashland, MA (US); Richard L. Damren, Marlborough, MA (US); Geoffrey L. Hodge, Sutton, MA (US)

Correspondence Address: Xcellerex, Inc.;Attention: Jacqueline Arendt, 170 Locke Drive, Marlborough, MA, 01752, US

(73) Assignee: Xcellerex, Inc., Marlborough, MA (US)

(21) Appl. No.: 12/011,493

(22) Filed: Jan. 25, 2008

(51) Int. Cl. B65B 31/00; B65D 30/10; B65D 30/00; C12M 1/34; B65B 67/12

(52) U.S. Cl. 53/434; 383/3; 383/37; 248/95; 435/287.1

(57) ABSTRACT

Systems and methods for containing and manipulating fluids, such as those involving collapsible bags and rigid containers that may be used as mixing systems, and as reactors for performing chemical, biochemical and/or biological reactions, are provided. A series of improvements and features for fluid containment systems such as containers including bag wrinkle removing systems, leak detection systems, and electromagnetic agitation systems are described.

### FIELD OF INVENTION

The present invention relates generally to systems for containing and manipulating fluids, and in certain embodiments, to systems and methods involving improvements to containers including bag wrinkle removing systems, leak detection systems, and electromagnetic agitation systems.

### BACKGROUND

A variety of vessels for manipulating fluids and/or for carrying out chemical, biochemical and/or biological reactions are available. For instance, biological materials (e.g., animal and plant cells) including, for example, mammalian, plant or insect cells and microbial cultures can be processed using bioreactors. Traditional bioreactors, which are typically designed as stationary vessels, or disposable bioreactors, many of which utilize sterile plastic containers, may be used. Although reaction systems and other fluid manipulating systems (e.g., mixing systems) are known, improvements to such systems would be beneficial.

### SUMMARY OF THE INVENTION

The present invention relates generally to systems for containing and manipulating fluids, and in certain embodiments, to systems and methods involving supported collapsible bags that may be used as bioreactors for performing chemical, biochemical and/or biological reactions contained therein. The subject matter of the present invention involves, in some cases, interrelated products, alternative solutions to a particular problem, and/or a plurality of different uses of one or more systems and/or articles. In the context of the present invention, it had been recognized that certain prior art systems, for example certain systems involving use of disposable plastic liners or bags in a support structure, suffer from problems caused by creases or other irregularities forming in the liner or bag upon filling of the bag/liner with liquid. Such creases undesirably can create “dead zones” that may lead, for example, to incomplete reaction, contamination or, in the base of bioreactors, necrosis. Certain such systems also suffer from the possibility of leakage from defects or damaged areas of the bag/liner. In these or other systems providing mixing via magnetically coupled impellers, improvements in the performance or controllability of mixing are also desirable and are provided by certain embodiments of the present invention.

In one embodiment, a vessel of the invention comprises a collapsible bag and a reusable support structure supporting and containing the collapsible bag. The vessel also includes a bladder or a compressible material positioned between an exterior wall of the collapsible bag and an interior wall of the support structure, the bladder or compressible material adapted to expand and/or contract so as to cause the collapsible bag to have a first configuration prior to expansion or contraction of the bladder or compressible material and a second configuration after expansion or contraction of the bladder or compressible material.

In another embodiment, a vessel of the invention comprises a collapsible bag and a reusable support structure comprising at least one wall portion that can be expanded or compressed so as to cause the collapsible bag to have a first configuration prior to expansion or compression of the wall portion and a second configuration after expansion or compression of the wall portion.

In one embodiment, a method of the invention comprises positioning a collapsible bag in a reusable support structure such that the reusable support structure contains and supports the collapsible bag, introducing a liquid into the collapsible bag, and changing a pressure of a fluid in a region between an exterior wall of the collapsible bag and an interior wall of the support structure.

In another embodiment, a vessel of the invention comprises a collapsible bag, a reusable support structure supporting and containing the collapsible bag, and a detector adapted to determine the presence of leakage of a fluid from the collapsible bag.

In another embodiment, a bioreactor system of the invention comprises a support structure and a rigid container or a collapsible bag positioned within the support structure. The rigid container or collapsible bag includes an impeller plate affixed to a lower portion of the rigid container or collapsible bag and an impeller hub mounted on the impeller plate, the impeller hub having at least one impeller blade and having at least one magnet. The rigid container or collapsible bag further includes a motor having a shaft, the motor being provided adjacent to or within the support structure, and a motor hub mounted on the motor shaft, the motor hub including at least one electromagnet, wherein upon mounting of the rigid container or collapsible bag within the support structure, the motor hub aligns with the impeller plate such that the electromagnet of the motor hub can drive the impeller hub when the motor shaft rotates. The impeller plate may optionally include a post. The bioreactor system may include one or more sensors for sensing one or more parameters of any materials in the rigid container or collapsible bag.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1 illustrates a vessel comprising a container contained within a support structure according to one embodiment of the invention;

FIG. 2 illustrates a vessel for carrying out fluid manipulations including biological, chemical, and biochemical processes, according to another embodiment of the invention;

FIG. 3 illustrates a bladder positioned in a vessel, according to another embodiment of the invention;

FIGS. 4-5 illustrate various leak detection systems, according to another embodiment of the invention;

FIG. 6 illustrates a magnetically-coupled impeller, according to another embodiment of the invention;

FIG. 7 illustrates an impeller including an electromagnetic drive, according to another embodiment of the invention;

FIG. 8 shows a mechanically-driven impeller, according to another embodiment of the invention; and

FIG. 9 shows an impeller magnetically coupled to an external motor, according to another embodiment of the invention.

### DETAILED DESCRIPTION

The present invention relates generally to systems for containing and manipulating fluids, and in certain embodiments, to systems and methods involving collapsible bags or liners and rigid containers that may be used as mixing systems, storage vessels, transfer vessels, or as reactors for performing chemical, biochemical or biological reactions. Certain embodiments of the invention involve a series of improvements and features for fluid containment systems, by, for example, providing vessels including wrinkle removing systems, leak detection systems, and/or electromagnetic agitation systems. For instance, the wrinkle removing systems can be configured to reduce or eliminate wrinkles in a collapsible bag or liner that may form when the bag or liner is filled with a liquid. Removal of wrinkles is often important as wrinkles create “dead spots” that can harbor undissolved solutes, cells, or chemicals, thereby reducing the speed of reaching homogeneity in mixing operations, reducing heat transfer efficiency due to lack of contact to the wall of a support structure that contains and supports the collapsible bag or liner, and/or creating regions that can trap cells and subject them to hypoxic or nutrient poor conditions causing necrosis, etc.

The leak detection systems described herein can be configured to detect leaks from a container and optionally notify the user of any leaks that may be formed before or during carrying out of a fluid manipulation process in a container. Leak detection is especially useful for vessels involving collapsible bags, liners, or other components containing fluid supported by reusable support structures, since leaks between walls of the vessels are otherwise difficult to detect and may cause catastrophic failure or release of hazardous agents.

In some vessels of the invention that include mixing systems, the system may include an electromagnetic agitation system that allows the user easier handling of components compared to systems that use permanent magnets. For instance, impellers and impeller hubs that include fixed magnets may be difficult to handle due to their strong attraction to one another, especially during assembly and disassembly of the components. This problem can be alleviated by using electromagnets which can be turned on and off by the user, thereby allowing the user to control the amount and period of attraction between the components.

Additional advantages and description of the above-mentioned systems are provided below.

The following documents are incorporated herein by reference: U.S. Provisional Patent Application Ser. No. 60/903,977, filed Feb. 28, 2007, entitled “Weight Measurements of Liquids in Flexible Containers,” by P. A. Mitchell, et al.; U.S. patent application Ser. No. 11/147,124, filed Jun. 6, 2005, entitled “Disposable Bioreactor Systems and Methods,” by G. Hodge, et al., published as U.S. Patent Application Publication No. 2005/0272146 on Dec. 8, 2005; International Patent Application No. PCT/US2005/020083, filed Jun. 6, 2005, entitled “Disposable Bioreactor Systems and Methods,” by G. Hodge, et al., published as WO 2005/118771 on Dec. 15, 2005; International Patent Application No. PCT/US2005/002985, filed Feb. 3, 2005, entitled “System and Method for Manufacturing,” by G. Hodge, et al., published as WO 2005/076093 on Aug. 18, 2005; U.S. patent application Ser. No. 11/818,901, filed Jun. 15, 2007, entitled, “Gas Delivery Configurations, Foam Control Systems, and Bag Molding Methods and Articles for Collapsible Bag Vessels and Bioreactors”; U.S. application Ser. No. 11/879,033, filed Jul. 13, 2007, entitled “Environmental Containment Systems”; U.S. Application Ser. No. 60/962,671, filed Jul. 30, 2007, entitled, “Continuous Perfusion Bioreactor System”; U.S. Application Ser. No. 60/903,977, filed Feb. 28, 2007, entitled “Weight Measurements of Liquids in Flexible Containers”; and a U.S. patent application filed on even date herewith, entitled, “Information Acquisition and Management Systems and Methods in Bioreactor Systems and Manufacturing Facilities”.

Although much of the description herein involves an exemplary application of the present invention related to bioreactors (and/or biochemical and chemical reaction systems), the invention and its uses are not so limited, and it should be understood that aspects of the invention can also be used in other settings, including those involving containment systems in general, as well as systems for containment and/or processing of a fluid in a container (e.g., mixing systems). It should also be understood that while many examples provided herein involve the use of collapsible bags, liners, or flexible containers, aspects of the invention can be integrated with systems involving non-collapsible bags, rigid containers, and other configurations involving liquid containment.

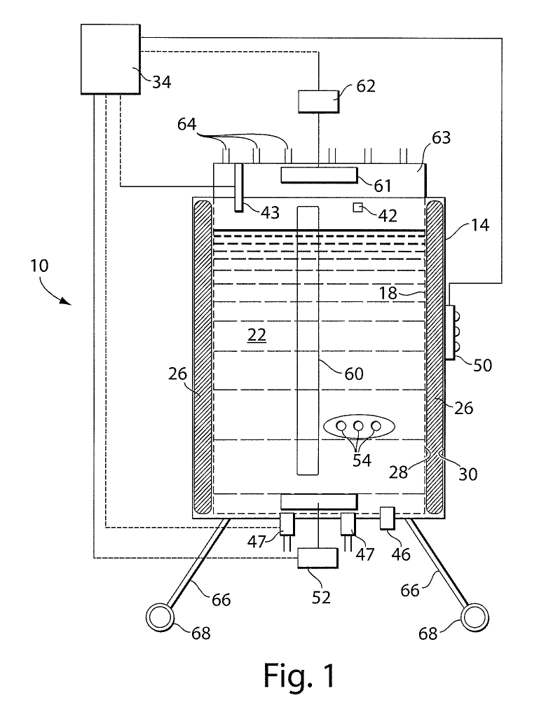

In one aspect, vessels configured to contain a volume of liquid are provided. In certain embodiments, the vessels are a part of a bioreactor system. For example, a non-limiting example of a vessel in the form of a bioreactor system including a container, such as a flexible container, is shown in the schematic diagram of FIG. 1. As shown in the embodiment illustrated in FIG. 1, vessel 10 includes a reusable support structure 14 (e.g., a stainless steel tank) that surrounds and contains a container 18. In some embodiments, the container is configured as a collapsible bag or liner (e.g., a polymeric bag) and may optionally include tubing, a magnetic drive pump, and/or a foam breaker. Additionally or alternatively, all or portions of the collapsible bag, liner or other container may comprise a substantially rigid material such as a rigid polymer, metal, or glass. In other embodiments, a rigid container is used in this configuration. The container may be disposable and may be configured to be easily removable from the support structure. In some embodiments, the container is irreversibly connected to the support structure.

If a collapsible bag is used, the collapsible bag may be constructed and arranged for containing a liquid 22, which may contain reactants, media, or other components necessary for carrying out a desired process such as a chemical, biochemical or biological reaction. The collapsible bag may also be configured such that liquid 22 remains substantially in contact only with the collapsible bag during use and not in contact with support structure 14. In such embodiments, the bag may be disposable and used for a single reaction or a single series of reactions, after which the bag is discarded. Because the liquid in the collapsible bag in such embodiments does not come into contact with the support structure, the support structure can be reused without cleaning. That is, after a reaction takes place in container 18, the container can be removed from the support structure and replaced by a second (e.g., disposable) container. A second reaction can be carried out in the second container without having to clean either the first container or the reusable support structure. If any liquid does come into contact with the reusable support structure due to leakage from the bag, in certain embodiments, one or more leak detection systems that are associated with vessel 10 detect the leak and notify the user so that appropriate measures can be taken.

In some embodiments, vessel 10 includes one or more wrinkle removal systems that reduce or eliminate wrinkles that may form in the collapsible bag when the bag is filled with a fluid and thereby pressed against the support structure. For instance, in the embodiment illustrated in FIG. 1, the wrinkle removal system includes one or more bladders 26 positioned between an exterior wall 28 of the collapsible bag and an interior wall 30 of the support structure. The bladder may be contracted (e.g., deflated) during or after introducing liquid 22 into the collapsible bag, effectively permitting the collapsible bag to stretch so as to remove or eliminate any wrinkles in the bag. Further description and additional examples of wrinkle removal systems are provided below.

Also shown in FIG. 1 are an optional inlet port 42 and optional outlet port 46, which can be formed in the container or reusable support structure and can facilitate more convenient introduction and removal of a liquid or gas from the container. The container may have any suitable number of inlet ports and any suitable number of outlet ports. For example, a plurality of inlet ports may be used to provide different gas compositions (e.g., via a plurality of spargers 47), or to allow separation of gases prior to their introduction into the container. These ports may be positioned in any suitable location with respect to container 18. For instance, for certain vessels including spargers, the container may include one more gas inlet ports located at a bottom portion of the container. Tubing may be connected to the inlet and outlet ports to form, e.g., delivery and harvest lines, respectively, for introducing and removing liquid from the container. Optionally, the container or support structure may include a utility tower 50, which may be provided to facilitate interconnection of one or more devices internal to the container or support structure with one or more pumps, controllers, or electronics (e.g., sensor electronics, electronic interfaces, and pressurized gas controllers) or other devices. Such devices may be controlled using a control system 34. The control system may also be used to send signals to and receive signals from a leak detection system and a wrinkle removal system.

For systems including multiple spargers, control system 34 may be operatively associated with each of the spargers and configured to operate the spargers independently of each other. This can allow, for example, control of multiple gases being introduced into the container.

In general, as used herein, a component of an inventive system that is “operatively associated with” one or more other components indicates that such components are directly connected to each other, in direct physical contact with each other without being connected or attached to each other, or are not directly connected to each other or in contact with each other, but are mechanically, electrically (including via electromagnetic signals transmitted through space), or fluidically interconnected so as to cause or enable the components so associated to perform their intended functionality.

The vessel may optionally include a mixing system such as an impeller 51, which can be rotated (e.g., about a single axis) using a motor 52 that can be external to the container. In some embodiments, as described in more detail below, the impeller and motor are magnetically coupled. The mixing system can be controlled by control system 34. Mixing systems are described in further detail below.

Additionally or alternatively, the vessel may include an antifoaming system such as a mechanical antifoaming device. As shown in the embodiment illustrated in FIG. 1, an antifoaming device may include, for example, an impeller 61 that can be rotated (e.g., magnetically) using a motor 62, which may be external to the container. The impeller can be used to collapse a foam contained in a head space 63 of the container. In some embodiments, the antifoaming system is in electrical communication with a sensor 43 (e.g., a foam sensor) via control system 34. The sensor may determine, for instance, the level or amount of foam in the head space or the pressure in the container, which can trigger regulation or control of the antifoaming system. In other embodiments, the antifoaming system is operated independently of any sensors.

The support structure and/or the container may also include, in some embodiments, one or more ports 54 that can be used for sampling, analyzing (e.g., determining pH and/or amount of dissolved gases in the liquid), or for other purposes. The support structure may also include one or more site windows 60 for viewing a level of liquid within the container. One or more connections 64 may be positioned at a top portion of the container or at any other suitable location. Connections 64 may include openings, tubes, and/or valves for adding or withdrawing liquids, gases, and the like from the container, each of which may optionally include a flow sensor and/or filter (not shown). The support structure may further include a plurality of legs 66, optionally with wheels 68 for facilitating transport of the vessel.

It should be understood that not all of the features shown in FIG. 1 need be present in all embodiments of the invention and that the illustrated elements may be otherwise positioned or configured. Also, additional elements may be present in other embodiments, such as the elements described herein. For example, in some embodiments, a vessel or one or more components of the vessel is associated with an “identifier”. The identifier(s) may be used to guide proper assembly of system components, verify that the system components are correctly assembled, protect against the use of counterfeit, improper or unauthorized components, etc. The identifier(s) may itself be “encoded with” information (i.e., carry or contain information, such as by use of an information carrying, storing, generating, or conveying device such as a radio frequency identification (RFID) tag or bar code) about the component including the identifier, or may not themselves be encoded with any information about the component, but rather may only be associated with information that may be contained in, for example, a database on a computer or on a computer readable medium. In the latter instance, detection of such an identifier can trigger retrieval and usage of the associated information from the database. Additional examples and uses of identifiers are described in more detail in a U.S. patent application filed on even date herewith, entitled, “Information Acquisition and Management Systems and Methods in Bioreactor Systems and Manufacturing Facilities”, which is incorporated herein by reference in its entirety.

In some embodiments, difficulty in accurately positioning the collapsible bag in the reusable support structure, e.g., because the shape of the collapsible bag may not match exactly the shape of the reusable support structure, and/or the construction of the collapsible bag (e.g., the presence of seams) may make it difficult to properly align the collapsible bag in the support structure, may be mitigated through the use of an inventive wrinkle removal system described herein. For instance, in some cases, without the inventive wrinkle removal systems, it is difficult or impossible to prevent folds and/or wrinkles from forming at a bottom portion of the collapsible bag because the collapsible bag may be constructed from flat sheet panels welded together to form a chamber, while the reusable support structure may have a curved bottom. Without the inventive wrinkle removal systems, once the folds or wrinkles have formed, they are difficult or impossible to remove since they will tend to be pressed up against the surface of the support structure.

As mentioned above, a vessel described herein (e.g., a bioreactor system) in certain embodiments includes one or more systems that can reduce or eliminate folds and/or wrinkles in a collapsible bag or liner. In some embodiments, a wrinkle removal system includes a bladder positioned between an exterior wall of the collapsible bag or liner and an interior wall of the support structure. The bladder can be expanded or contracted, e.g. pneumatically, to effectively modify the internal volume and/or shape of the support structure, thereby modifying the configuration of the collapsible bag or liner.

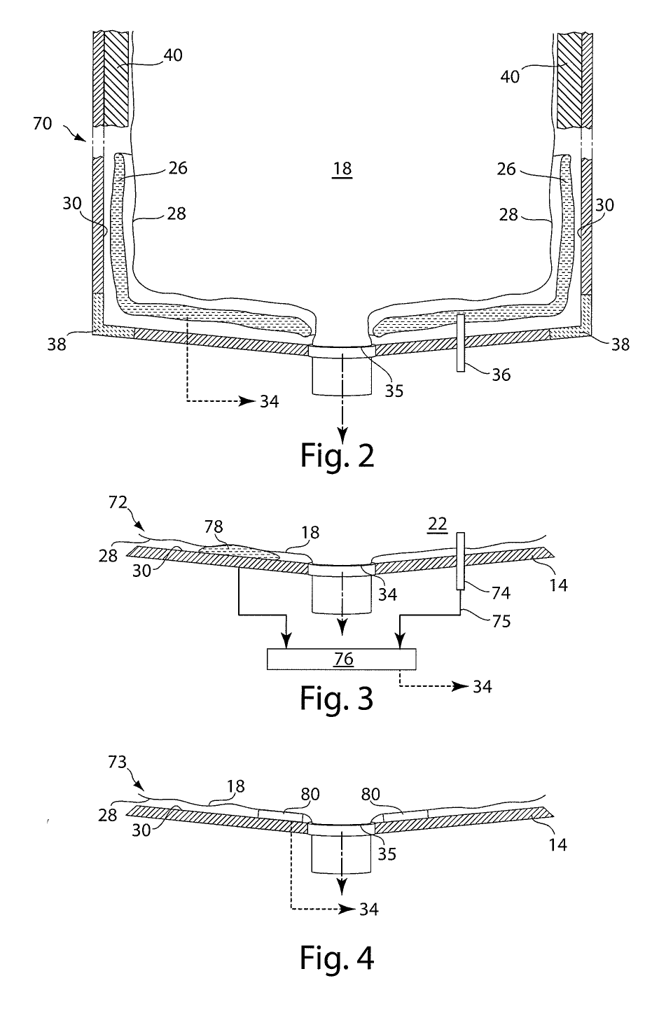

As shown in the embodiment illustrated in FIG. 2, vessel 70 includes one or more bladders 26 positioned at bottom and side portions of collapsible bag 18, which is contained in and supported by reusable support structure 14. The bladder(s) may be filled with a liquid or a gas so as to have a desired volume and/or shape for supporting the collapsible bag. The collapsible bag can then be filled partially or completely such that at least a portion of a wall of the collapsible bag contacts or is pressed up against a surface of the bladder. Sometimes, as the collapsible bag is filled, portions of the collapsible bag wall may not lay flat against the expanded bladder. This can cause folds or wrinkles to form at the wall portion of the collapsible bag, which can negatively effect the materials or process being performed inside the container. When bladder 26 is contracted, however, the collapsible bag can further expand to a configuration that has fewer wrinkles or folds in one or more wall portions of the collapsible bag. Contraction can take place, for example, by removing all or portions of the contents in the bladder (e.g., by deflating or draining the contents from the bladder). This process can effectively stretch the collapsible bag to remove some or all of the wrinkles or folds in the bag.

The bladder may have any suitable volume and shape that can be expanded and/or contracted so as to cause the collapsible bag to have a first configuration prior to expansion or contraction of the bladder and a second configuration after expansion or contraction of the bladder. The bladder may have a shape that matches the configuration of the reusable structure and/or the collapsible bag and, in some embodiments, may conform to the shape of the reusable support structure and/or the shape of the collapsible bag. For example, as illustrated in FIG. 2, bladder 26 may be designed such that it extends from an outer side wall 28 of the collapsible bag to drain hole 35 of the collapsible bag and support structure.

It should be understood that bladder 26 can be positioned at any suitable position with respect to collapsible bag 18 and reusable support structure 14. For instance, in some embodiments, one or more bladders completely surround an outer wall 28 of the collapsible bag or is completely circumscribed by an inner wall 30 of the reusable support structure. In other embodiments, one or more bladders is contiguous with only a portion of the perimeter of the collapsible bag or the reusable support structure. For example, the bladder may be configured for contact with all or a portion of a bottom portion, a top portion, or side portion of the collapsible bag or reusable support structure. In some cases, an array of bladders may be positioned around the collapsible bag and within the reusable support structure.

The bladder(s) may be positioned within the reusable support structure using any suitable positioning or attachment technique. In some embodiments, the bladder is removably or irreversibly attached to the collapsible bag. The bladder may be removably attached to inner surface 30 of the reusable support structure using, for example, adhesives, magnetic interactions, pressure (e.g., pressed up against the inner surface of the support structure when the bladder is expanded), and the like. The reusable support structure may be lined with the bladder after or prior to introducing the collapsible bag into the support structure. In another embodiment, the bladder is fabricated together with the collapsible bag (for example, by injection or blow molding), e.g., such that the interior of the bladder is not in fluid communication with the interior of the collapsible bag. This irreversible attachment of the bladder and the collapsible bag can facilitate introduction and removal of the bladder and bag into and from the support structure, in some embodiments, since there are fewer pieces to position and align. In another embodiment, the collapsible bag and the bladder are two separate entities that can be associated with one another using, for example, adhesives, pressure, magnetic interactions, and the like, prior to introducing the units into the reusable support structure. In yet another embodiment, the collapsible bag is first inserted into the reusable support structure, after which the bladder can be positioned between the collapsible bag and the support structure.

Any suitable numbers of bladders can be associated with a collapsible bag and/or support structure. Where more than one bladders are used, the bladders may be operated independently of one another. For instance, the bladders may be controlled independently to cause each of the bladders to expand or contract depending on it's location with respect to the collapsible bag and/or support structure, the amount of fluid and/or pressure in the collapsible bag, etc. The bladders may be self-expandable/collapsible in some cases, for example, by using air to inflate or deflate the bladder. In some cases, the bladders are associated with a sensor, e.g., a pressure sensor, which can be used to measure the internal pressure of the bladder. The bladder may be programmed to maintain a constant pressure, or to operate within a range of pressures. For example, prior to the collapsible bag being filled with a fluid, the bladder may have a first internal pressure. As the collapsible bag is filled with fluid, the pressure may be exerted against the bladder, thereby increasing the internal pressure of the bladder. This increase in pressure may be detected by the sensor, and in response, the bladder may self-contract (e.g., self-deflate) until the internal pressure of the bladder reaches the first internal pressure. Meanwhile, the reduction of volume inside the bladder can cause a change in configuration of the collapsible bag. The volume of the collapsible bag may, for example, effectively increase due to the collapse of the bladder and/or the number and/or size of the wrinkles or folds in the collapsible bag may be decreased.

In some cases, the bladder is specifically adapted to expand or contract upon the operation of one or more other components of the collapsible bag and/or support structure such as a sensor, heater, upon opening or closing of a port, and the like.

Bladder 26 may include one or more ports (e.g., port 36) for introducing or removing a substance, such as a gas, liquid, gel, or a solid, from the bladder. The port may be accessible from an exterior portion of the support structure in some embodiments. The port may have any suitable size and configuration, and may be made from any suitable material. In some cases, the port and/or the material used to form the bladder includes a self-sealing material such as a silicone.

In some embodiments, the collapsible bag is designed to have a shape and volume substantially similar to that of the support structure. Thus, when the bladder is contracted or deflated fully, the collapsible bag is only separated from the support structure by the thin layer(s) forming the bladder. Such an embodiment may be useful for facilitating heat exchange between the contents inside the collapsible bag and the support structure and/or an environment outside the support structure, as heat can dissipate from the collapsible bag to the support structure via the bladder. All or portions of the bladder may be formed from a thermally conductive material to facilitate heat transfer, as described in more detail below. In other embodiments, the collapsible bag is designed to have a volume less than the volume of the support structure absent the bladder (e.g., when the collapsible bag is fully expanded), but greater than the internal volume of the support structure in the presence of the bladder, when in an expanded configuration. Thus, when the collapsible bag is fully extended, the bladder may still be partially inflated. This configuration of the bladder can prevent the bag from extending to the inner surface of the support structure. Such an embodiment may be useful, for example, for insulating the collapsible bag from the support structure.

Certain embodiments of the invention include a support structure comprising at least one wall portion that can expand and/or contract so as to cause a collapsible bag to have a first configuration prior to expansion or contraction of the wall portion, and a second configuration after expansion or contraction of the wall portion. For example, in one embodiment the support structure includes one or more adaptable portions that can allow at least a portion of the support structure to expand (or contract), e.g., upon filling or emptying of the collapsible bag. This can allow the collapsible bag to have a larger (or smaller) volume than would be the case without adaptable portion(s) and/or can effectively stretch the collapsible bag to remove or reduce any folds and/or wrinkles in the bag. As illustrated in the exemplary embodiment shown in FIG. 2, the adaptable portion of the wall may comprise an expandable seam 38, which may be positioned at a corner, an edge, a surface (e.g., a face) of the support structure, or at any other suitable position. The adaptable portion of the wall may include, in some embodiments, a hinge, a telescoping joint or surface, a flexible material (e.g., a flexible polymer), or the like. The adaptable portion may expand or contract automatically, e.g., based on the volume of fluid and/or pressure inside the collapsible bag, or the adaptable portion may be controlled manually by a user or by an automated control system.

In certain embodiments of the invention, the support structure includes a compressible material whose use can reduce or remove wrinkles in a collapsible bag. As shown in the embodiment illustrated in FIG. 2, compressible material 40 may comprise, for example, an elastomeric material, a spring plate or other spring-biased member(s), or a composite that can allow the collapsible bag to have a first configuration prior to expansion or contraction of the compressible material, and a second configuration after expansion or contraction of the compressible material. In some cases, compressible material 40 is a foam or other porous structure that can contain a higher amount of gas (e.g., air) in the expanded state than in the contracted or compressed state.

In one embodiment, the compressible material has a first configuration prior to introducing a fluid into the collapsible bag and a second configuration after introducing a fluid into the collapsible bag. For example, as the collapsible bag is being filled, the outward pressure of the fluid in the collapsible bag can cause all or portions of the compressible material to compress. This can effectively allow the collapsible bag to stretch, thereby reducing or eliminating folds or wrinkles in the walls of the collapsible bag.

Accordingly, in one particular embodiment, a vessel of the invention comprises a collapsible bag and a reusable support structure supporting and containing the collapsible bag. The vessel further includes a bladder or a compressible material positioned between an exterior wall of the collapsible bag and an interior wall of the support structure. The bladder or compressible material is adapted to expand and/or contract so as to cause the collapsible bag to have a first configuration prior to expansion or contraction of the bladder or compressible material and a second configuration after expansion or contraction of the bladder or compressible material. In another embodiment, a vessel of the invention comprises a collapsible bag and a reusable support structure comprising at least one wall portion that can be expanded or compressed so as to cause the collapsible bag to have a first configuration prior to expansion or compression of the wall portion and a second configuration after expansion or compression of the wall portion.

In some cases, wrinkles and/or folds in walls of the collapsible bag can be removed by creating a vacuum between an outer wall of the collapsible bag and an interior wall of the reusable support structure. The vacuum can cause any air pockets that are formed as a result of the wrinkles and/or folds to be removed, thereby permitting the wall of the collapsible bag to lay flat against a greater portion of, or substantially the entirety of, the interior wall of the reusable support structure. A source of vacuum may be engaged and in fluid communication with a space between an outer wall of the collapsible bag and an interior wall of the reusable support structure at any suitable location along the support structure via tubing or other suitable means. In some cases, the support structure includes ports (not shown) that are adapted for connection to a source of vacuum. The ports can be positioned at various locations around the support structure. Application of a vacuum can take place before, after, or during introduction of a liquid or other processing material into the collapsible bag.

One particular method of the invention includes positioning a collapsible bag in a reusable support structure such that the reusable support structure contains and supports the collapsible bag, introducing a liquid into the collapsible bag, and changing a pressure of a fluid in a region between an exterior wall of the collapsible bag and an interior wall of the support structure. In one embodiment, the change in pressure involves creating a vacuum between the exterior wall of the collapsible bag and the interior wall of the support structure. In another embodiment, the change in pressure involves increasing or decreasing a positive pressure (e.g., inflating or deflating a bladder) between the exterior wall of the collapsible bag and the interior wall of the support structure.

Certain embodiments of the invention include a vessel comprising a detector adapted to determine the presence of any liquid that leaks from the collapsible bag. Detection of leaks is often difficult in conventional vessels, especially vessels that include a collapsible bag supported by a reusable support structure. Advantageously, it is desirable to detect leakage while the leak(s) is small so that appropriate measures can be taken before the leakage increases.

In one embodiment, a vessel of the invention includes a detector that is constructed and arranged to detect an electrical conductance or impedance change between the fluid inside the container and the reusable support structure. If the collapsible bag is intact and does not have any leaks, no change in electrical signal will be detected. An example of such a system is shown in the embodiment illustrated in FIG. 3. Vessel 72 includes a conductance probe 74 that is in contact with a fluid 22 inside a collapsible bag 18. The probe can be inserted or embedded in a wall of the collapsible bag, for example. In one embodiment, the probe acts as a ground and is electrically insulated from the wall of support structure 14. Measurements may be made by applying a small electrical potential to the fluid contained in the collapsible bag via the probe. If the collapsible bag leaks, an impedance or conductivity detector that is connected to the probe and the wall of the support structure will detect a closing of an electrical circuit by the conductive fluid contents of the bag or a change in resistance between the fluid and the wall of the support structure. For instance, in the example illustrated in FIG. 3, probe 74 may be in electrical communication with detector 76 (e.g., an impedance or conductivity detector), which is configured to detect a change in the electrical properties (e.g., voltage, current, resistance, or impedance) due to the presence of any leakage of fluid from the collapsible bag. In one embodiment, electrical circuit 75 is completed by the presence of a leaked fluid 78 (e.g., an electrically-conductive material) positioned between outer wall 28 of the collapsible bag and inner wall 30 of the support structure, all or a portion of which may be conductive or semi-conductive. In another embodiment, fluid 78 causes current flow to increase and impedance between fluid 22 and a wall of the support structure to drop.

Another leakage detection system is shown in the embodiment illustrated in FIG. 4. Leakage detection system 73 includes one or more moisture detectors 80 that are positioned between an outer wall 28 of collapsible bag 18 and an inner wall 30 of reusable support structure 14. As illustrated in FIG. 4, moisture detector 80 may be positioned at or near an opening 35 of the collapsible bag and/or the support structure. By positioning the detector at this location, any fluid near the opening of the collapsible bag or reusable support structure can be detected before the fluid leaks out of the system.

A vessel of the invention may include one or more detectors 76 and/or 80 positioned at various locations in the system. Detectors 76 and 80 may make measurements continuously, periodically, and/or in some cases, in response to certain events, e.g., upon the introduction of a liquid into the container. Signals from detectors 76 and/or 80 may sound an alarm, be sent to a control unit 34 to notify the user of the presence or absence of leakage, and/or may activate measures that can control or eliminate leaking (e.g., activation of a self-sealing material). In some cases, a signal can cause all or portions of the system to shut down.

Detectors for determining leaks and/or moisture are known and can be incorporated into systems described herein by methods known to those of ordinary skill in the art in conjunction with the description provided herein. Non-limiting examples of leak detectors include those described in U.S. Pat. Nos. 6,229,229; 6,873,263; and 7,292,155, which are incorporated herein by reference. In addition, it should be understood that any suitable change in physical, electrical, optical, etc. properties can be measured and used to indicate the presence of leakage and/or moisture in vessels described herein. Non-limiting examples of parameters that can be monitored to indicate leakage and/or moisture include changes in color, absorbance, turbidity, opacity, conductance, impedance, resistance, pressure, volume, and temperature.

Various aspects of the present invention are directed to a vessel including a container such as a collapsible bag. “Flexible container”, “flexible bag”, or “collapsible bag” as used herein, indicates that the container or bag is unable to maintain its shape and/or structural integrity when subjected to the internal pressures (e.g., due to the weight and/or hydrostatic pressure of liquids and/or gases contained therein expected during operation) without the benefit of a separate support structure. The collapsible bag may be made out of inherently flexible materials, such as many plastics, or may be made out of what are normally considered rigid materials (e.g., glass or certain metals) but having a thickness and/or physical properties rendering the container as a whole unable to maintain its shape and/or structural integrity when subjected to the internal pressures expected during operation without the benefit of a separate support structure. In some embodiments, collapsible bags include a combination of flexible and rigid materials; for example, the bag may include rigid components such as connections, ports, supports for a mixing and/or antifoaming system, etc.

The container (e.g., collapsible bag) may have any suitable size for containing a liquid. For example, the container may have a volume between 0.1-5 L, 1-40 L, 40-100 L, 100-200 L, 200-300 L, 300-500 L, 500-750 L, 750-1,000 L, 1,000-2,000 L, 2,000-5,000 L, or 5,000-10,000 L. Volumes greater than 10,000 L are also possible.

In some embodiments, the container (e.g., collapsible bag) is formed of a suitable flexible material. The flexible material may be one that is USP Class VI certified, e.g., silicone, polycarbonate, polyethylene, and polypropylene. Non-limiting examples of flexible materials include polymers such as polyethylene (e.g., linear low density polyethylene and ultra low density polyethylene), polypropylene, polyvinylchloride, polyvinyldichloride, polyvinylidene chloride, ethylene vinyl acetate, polycarbonate, polymethacrylate, polyvinyl alcohol, nylon, silicone rubber, other synthetic rubbers and/or plastics. As noted above, portions of the flexible container may comprise a substantially rigid material such as a rigid polymer (e.g., high density polyethylene), metal, and/or glass (e.g., in areas for supporting fittings, etc.). In other embodiments, the container is a substantially rigid material. Optionally, all or portions of the container may be optically transparent to allow viewing of contents inside the container. The materials or combination of materials used to form the container may be chosen based on one or more properties such as flexibility, puncture strength, tensile strength, liquid and gas permeabilities, opacity, and adaptability to certain processes such as blow molding, injection molding, or spin cast molding (e.g., for forming seamless collapsible bags). The container may be disposable in some cases.

The container (e.g., collapsible bag) may have any suitable thickness for holding a liquid and may be designed to have a certain resistance to puncturing during operation or while being handled. For instance, the walls of a container may have a total thickness of less than or equal to 250 mils (1 mil is 25.4 micrometers), less than or equal to 200 mils, less than or equal to 100 mils, less than or equal to 70 mils (1 mil is 25.4 micrometers), less than or equal to 50 mils, less than or equal to 25 mils, less than or equal to 15 mils, or less than or equal to 10 mils. In some embodiments, the container includes more than one layer of material that may be laminated together or otherwise attached to one another to impart certain properties to the container. For instance, one layer may be formed of a material that is substantially oxygen impermeable. Another layer may be formed of a material to impart strength to the container. Yet another layer may be included to impart chemical resistance to fluid that may be contained in the container. One or more layers of the container may include a thermally-conductive material to facilitate heat transfer to and from the interior of the container to an environment outside of the container.

It should be understood that a container, liner, or other article described herein (e.g., a bladder) may be formed of any suitable combinations of layers and that the invention is not limited in this respect. The article (e.g., collapsible bag) may include, for example, 1 layer, greater than or equal to 2 layers, greater than or equal to 3 layers, or greater than equal to 5 layers of material(s). Each layer may have a thickness of, for example, less than or equal to 200 mils, less than or equal to 100 mils, less than or equal to 50 mils, less than or equal to 25 mils, less than or equal to 15 mils, less than or equal to 10 mils, less than or equal to 5 mils, or less than or equal to 3 mils, or combinations thereof.

In one set of embodiments, the container or liner is seamless. The container may be, for example, a seamless collapsible bag or a seamless rigid (or semi-rigid) container. Many existing collapsible bags are constructed from two sheets of a plastic material joined by thermal or chemical bonding to form a container having two longitudinal seams. The open ends of the sheets are then sealed using known techniques and access apertures are formed through the container wall. During use, collapsible bags having seams can cause the formation of crevices at or near the seams where fluids or reagents contained therein are not thoroughly mixed. In certain embodiments involving, for example, the use of collapsible bags for performing a chemical, biochemical and/or biological reaction, unmixed reagents can cause a reduction in yield of a desired product. The presence of the seams in a collapsible bag can also result in the inability of the collapsible bag to conform to the shape of a reusable support structure that may support the bag. By using collapsible bags without any seams joining two or more flexible wall portions of the bag, however, the problems of mixing and conformity may be avoided or reduced. Seamless collapsible bags can also be used together with bladders or other wrinkle removal systems of the invention.

In certain embodiments, seamless collapsible bags can be made specifically to fit a particular reusable support structure having a unique shape and configuration. Substantially perfect-fitting collapsible bags can be used, for example, as part of a bioreactor system or a biochemical or chemical reaction system. Seamless rigid or semi-rigid containers may also be beneficial in some instances. Additional description of seamless containers can be found in U.S. patent application Ser. No. 11/818,901, filed Jun. 15, 2007, entitled, “Gas Delivery Configurations, Foam Control Systems, and Bag Molding Methods and Articles for Collapsible Bag Vessels and Bioreactors”, which is incorporated herein by reference.

In certain embodiments, a collapsible bag that does not include any seams joining two or more flexible wall portions of the collapsible bag (i.e., a seamless collapsible bag) has a certain volume for containing a liquid. The seamless collapsible bag may have a volume of, for example, at least 0.1 L, at least 1 L, at least 10 liters, at least 20 liters, at least 40 liters, at least 50 liters, at least 70 liters, at least 100 liters, at least 150 liters, at least 200 liters, at least 300 liters, at least 500 liters, at least 700 liters, or at least 1,000 liters. Seamless collapsible bags may also have volumes greater than 1,000 liters (e.g., 1,000-5,000 liters or 5,000-10,000 liters) as needed. In some embodiments, the collapsible bag is positioned in a reusable support structure for surrounding and containing the flexible container.

In one embodiment, a seamless collapsible bag is formed in a process in which the bag liner (e.g., the flexible wall portions of the bag), as well as one or more components such as a component of an agitator/mixer system (e.g., a shaft and/or a support base), port, bladder, etc. is cast from one continuous supply of a polymeric precursor material. In some cases, the casting may occur without hermetically sealing, e.g., via welding. Such a seamless collapsible bag may allow the interior liquid or other product to contact a generally even surface, e.g., one which does not contain substantial wrinkles, folds, crevices, or the like. In addition, in some cases, the collapsible bag complementarily fits within a support structure when installed and filled with a liquid or product. The seamless collapsible bag may also have a generally uniform polymeric surface chemistry which may, for example, minimize side reactions. Collapsible bags involving more than one polymeric precursor materials can also be formed.

Seamless collapsible bags can be created by a variety of methods. In one embodiment, a seamless collapsible bag is formed by injecting liquid plastic into a mold that has been pre-fitted with components such as ports, connections, supports, and rigid portions configured to support a mixing system (e.g., a shaft and/or a base) that are subsequently surrounded, submerged, and/or embedded by the liquid plastic. The component may be a rigid component, e.g., one that can substantially maintain its shape and/or structural integrity during use. Any suitable number of components (e.g., at least 1, 2, 5, 10, 15, etc.) can be integrated with containers (e.g., collapsible bags) using methods described herein. The mold may be designed to form a collapsible bag having the shape and volume of the mold, which may have a substantially similar shape, volume, and/or configuration of a reusable support structure.

In one embodiment, the container is formed by using an embedded component/linear molding (ECM) technique. In one such technique, rigid or pre-made components such as tube ports, agitator bases, etc. are first positioned in the mold. A polymer or polymer precursor used to form a container (e.g., a seamless collapsible bag) may be introduced (e.g., in a melt state) via a polymer fabrication technique such as those described below. In some cases, a component or a portion of the component is partially melted by the polymer precursor, allowing the component to form a continuous element with the container. That is, the component can be joined (e.g., fused) with one or more wall portions of the container (e.g., flexible wall portions of a collapsible bag) to form a single, integral piece of material without seams. In other cases, components are designed with thinner portions that can be melted with a polymer precursor (e.g., in the melt state) during formation of a container.

Accordingly, one method of joining together a wall portion of a container and at least a portion of a functional component during formation of the container within a mold includes melting the portion of the functional component during the joining step. The wall portion of the container may have a first thickness and the portion of the functional component may have a second thickness, the thicknesses being within, for example, less than 100%, 80%, 60%, 40%, 20%, 10%, or 5% of each other, relative to the larger of the first and second thicknesses.

In another embodiment, the container may be formed using a continuous component/liner molding (CCM) technique. In one such technique, a collapsible bag or other container is cast de novo from a polymer or polymer precursor stream. The polymer or polymer precursor used to form the seamless collapsible bag is introduced via a polymer fabrication technique such as those described below. Components can be introduced into the flexible container by using mandrels, barriers, baffles, and the like to direct the polymer precursor to form functional components of a liquid containment system such as tube ports and agitator bases as, for example, one continuous polymer. After setting or curing of the polymer or polymer precursor, the mandrels, barriers, etc., may be withdrawn.

Combinations of these and/or other techniques may also be used in other embodiments. For instance, in some cases, different polymer formulations (such as low molecular weight polyethylene, high molecular weight polyethylene, polypropylene, silicone, polycarbonate, polymethacrylate, combinations thereof or precursors thereof) can be simultaneously injected into regions of the mold designed to form a more rigid structure such as tubing or sensor ports, agitation systems, etc.

In one particular embodiment, a method involves introducing a first polymer or polymer precursor into a mold comprising a shape configured to mold a collapsible bag having a volume of at least 10 mL, IL, 40 L, 100 L, or 1,000 L, etc. The mold may further comprise a shape configured to mold a component of a mixing and/or antifoaming system such as a shaft and/or base configured to support an impeller. The method may also include introducing a second polymer or polymer precursor into the mold to mold the component of the mixing system. Accordingly, the component of the mixing system and the collapsible bag may be joined without welding using methods described herein. In some instances, the first and second polymers or polymer precursors are introduced simultaneously. The first and second polymers or polymer precursors may be the same in some embodiments, or different in other embodiments. Such a method can be used to form, for example, base plates for mixing/agitation systems, antifoaming systems, or other components. In other embodiments, a number of polymers can be introduced into the mold (e.g., simultaneously) to form containers with multiple components.

As mentioned, a polymer or polymer precursor may be introduced into a mold to form a container such as a collapsible bag (e.g., a seamless collapsible bag) using any suitable technique. For instance, in one embodiment, the collapsible bag may be fabricated via a spin casting process. For example, during spin casting, a mold may be spun during injection of the polymer or polymeric precursor to deposit a uniform coating of plastic on the mold surface. In another embodiment, the collapsible bag is fabricated via an injection molding process. For instance, the polymeric precursor may be pumped into the space between an inner mold and the outer mold. In yet another embodiment, the collapsible bag can be fabricated via a blow molding process. The polymer may be deposited, for example, via a gas injection, to expand the polymer against the mold surface. In yet another embodiment, a combination of these and/or other techniques may be used. Those of ordinary skill in the art will be familiar with polymer processing techniques such as spin casting, injection molding, and/or blow molding, and will be able to use such techniques to prepare suitable collapsible bags or other containers. Such techniques can also be used to form bladders or other components of the invention.

Although many embodiments herein describe seamless collapsible bags, in some embodiments, collapsible bags or other containers described herein can be fabricated with seams between flexible wall portions of the container. In other embodiments, collapsible bags or other containers can be fabricated with seams between a component and one or more flexible wall portions of the container. The act of joining two or more wall portions or a wall portion and a portion of a component may be achieved by methods such as welding (e.g., heat, welding and ultrasonic welding), bolting, use of adhesives, fastening, or other attaching techniques. Combination of seams and seamless connections can also be fabricated.

It should also be understood that while many of the methods described herein refer to fabrication of collapsible bags, the methods may also be applied to rigid containers or components of vessels. The methods described herein used to form containers such as collapsible bags (e.g., bags with or without seams) may be adapted to include components of various sizes. For instance, although the flexible wall portions of a collapsible bag may having a thickness of, for example, less than or equal to 100 mils, less than or equal to 70 mils, less than or equal to 50 mils, less than or equal to 25 mils, less than or equal to 15 mils, or less than or equal to 10 mils, a component to be incorporated with the container may have a thickness or a height of, for example, greater than 0.5 mm, greater than 1 cm, greater than 1.5 cm, greater than 2 cm, greater than 5 cm, or greater than 10 cm. In some cases, the component has at least one cross-sectional dimension (e.g., a height, length, width, or diameter) of, for example, greater than 0.5 mm, greater than 1 cm, greater than 1.5 cm, greater than 2 cm, greater than 5 cm, greater than 10 cm, greater than 15 cm, greater than 20 cm, greater than 25 cm, or greater than 30 cm. In certain embodiments, the thickness of a collapsible bag (or other container) and the thickness of a portion of a component to be joined (e.g., fused) with the collapsible bag are within 30%, 20%, 15%, 10% or 5% of each other (relative to the thickest portion). This matching of thicknesses can aid joining (e.g., melting, welding, etc.) of the materials, as described in more detail below.

Components that are integrated with collapsible bags or other containers may be formed in any suitable material, which may be the same or a different material from that of the bag or container. For instance, in one embodiment, a container is formed in a first polymer and a component is formed in a second polymer that is different (e.g., having a different composition, molecular weight, and/or chemical structure, etc.) from the first polymer. Those of ordinary skill in the art will be familiar with material processing techniques and will be able to use such techniques in the methods described herein to choose suitable materials and combinations of materials.

In some embodiments, components that are integrated with collapsible bags or other containers using methods described herein are formed from one or more materials that is/are USP Class VI certified, e.g., silicone, polycarbonate, polyethylene, and polypropylene or, alternatively, are formed from one or more non-certified materials. Non-limiting examples of materials that can be used to form components include polymers such as polyethylene (e.g., low density polyethylene and high density polyethylene), polypropylene, polyvinylchloride, polyvinyldichloride, polyvinylidene chloride, ethylene vinyl acetate, polyvinyl alcohol, polycarbonate, polymethacrylate, nylon, silicone rubber, other synthetic rubbers and/or plastics, and combinations thereof. Ceramics, metals, and magnetic materials can also be used to form all or portions of a component. In some embodiments, all or portions of a component are rigid; in other embodiments, all or portions of a component are flexible. The material(s) used to form a component may be chosen based on, for example, the function of the component and/or one or more properties such as compatibility with the container, flexibility, tensile strength, hardness, liquid and gas permeabilities, and adaptability to certain processes such as blow molding, injection molding, or spin cast molding.

In certain embodiments, especially in certain embodiments involving fluid manipulations or carrying out a chemical, biochemical and/or biological reaction in a container (e.g., a collapsible bag), the container is substantially closed, e.g., the container is substantially sealed from the environment outside of the container except, in certain embodiments, for one or more inlet and/or outlet ports that allow addition to, and/or withdrawal of contents from, the container. If a collapsible bag is used, it may be substantially deflated prior to being filled with a liquid, and may begin to inflate as it is filled with liquid. In other embodiments, aspects of the invention can be applied to opened container systems.

In some cases, fluids may be introduced and/or removed from a vessel via inlet ports and/or outlet ports. The vessel may be a part of a reactor system for performing a biological, biochemical, or chemical reaction. As mentioned, a container (e.g., a collapsible bag), which may be part of the vessel, may have any suitable number of inlet ports and any suitable number of outlet ports. In some cases, pumps, such as disposable pumps, may be used to introduce a gas or other fluid into the container, e.g., via an inlet port, and/or which may be used to remove a gas or other fluid from the container, e.g., via an outlet port. For instance, a magnetically-coupled pump may be created by encasing a disposable magnetic impeller head in an enclosure with inlet(s) and outlet(s) that achieves fluid pumping. Flexible blades may be used to enhance pumping or provide pressure relief. In another embodiment, pumping of fluids, gas and/or powder may be achieved without pump heads and/or pump chambers by sequentially squeezing, for example, an electromechanical-polymeric tube that effectively achieves “peristalsis.” One way valves in the tube may optionally be used, which may aid in the prevention of backflow.

In some embodiments, a support structure, for example, support structure 14 as shown in FIG. 1, can be used to surround and contain container 18. The support structure may have any suitable shape able to surround and/or contain the container. In some cases, the support structure is reusable. The support structure may be formed of a substantially rigid material. Non-limiting examples of materials that can be used to form the reusable support structure include stainless steel, aluminum, glass, resin-impregnated fiberglass or carbon fiber, polymers (e.g., high-density polyethylene, polyacrylate, polycarbonate, polystyrene, nylon or other polyamides, polyesters, phenolic polymers, and combinations thereof. The materials may be certified for use in the environment in which it is used. For example, non-shedding materials may be used in environments where minimal particulate generation is required.

In some embodiments, the reusable support structure may be designed to have a height and diameter similar to standard stainless steel bioreactors (or other standard reactors or vessels). The design may also be scaleable down to small volume bench reactor systems. Accordingly, the reusable support structure may have any suitable volume for carrying out a desired chemical, biochemical and/or biological reaction. In many instances, the reusable support structure has a volume substantially similar to that of the container. For instance, a single reusable support structure may be used to support and contain and single container having a substantially similar volume. In other cases, however, a reusable support structure is used to contain more than one container. The reusable support structure may have a volume between, for example, 0.1-5 L, 1-100 L, 100-200 L, 200-300 L, 300-500 L, 500-750 L, 750-1,000 L, 1,000-2,000 L, 2,000-5,000 L, or 5,000-10,000 L. Volumes greater than 10,000 L are also possible.

In other embodiments, however, a vessel does not include a separate container (e.g., collapsible bag) and support structure, but instead comprises a self-supporting disposable container. The container may be, for example, a plastic vessel and, in some cases, may include an agitation system integrally or removably attached thereto. The agitation system may be disposable along with the container. In one particular embodiment, such a system includes an impeller welded or bolted to a polymeric container. It should therefore be understood that many of the aspects and features of the vessels described herein with reference to a container and a support structure (for example, a seamless container, a sparging system, an antifoaming device, a bladder, a wrinkle removal system, a leak detection system, a heat-conduction system, an electromagnetic mixing system, etc.), are also applicable to a self-supporting disposable container.

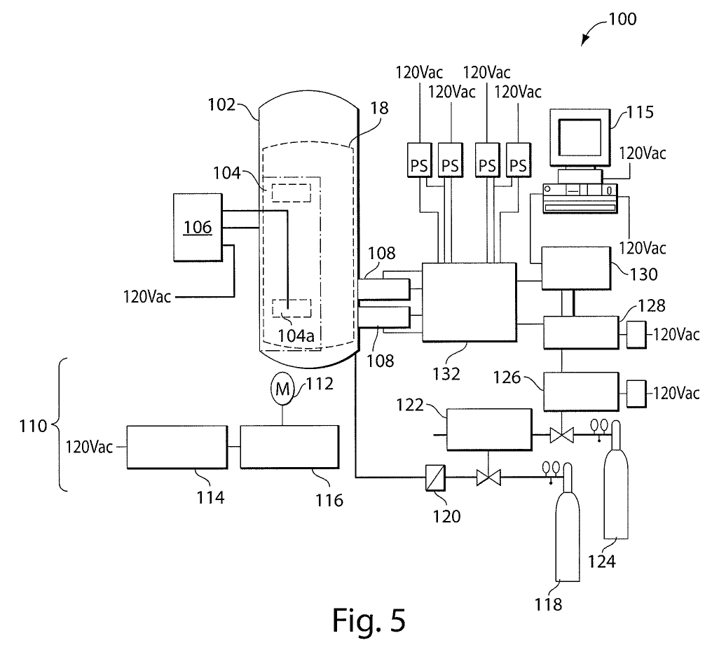

In some embodiments, a container, such as container 18 shown in FIG. 5, may include various sensors and/or probes for controlling and/or monitoring one or more process parameters inside the container such as, for example, temperature, pressure, pH, dissolved oxygen (DO), dissolved carbon dioxide (DCO₂), mixing rate, and gas flow rate. The sensor may also be an optical sensor in some cases.

In some cases, process control may be achieved in ways which do not compromise the sterile barrier established by a container (e.g., collapsible bag). For example, gas flow may be monitored and/or controlled by a rotameter or a mass flow meter upstream of an inlet air filter. In another embodiment, disposable optical probes may be designed to use “patches” of material containing an indicator dye which can be mounted on the inner surface of the container and read through the wall of the container via a window in the reusable support structure. For example, dissolved oxygen, pH, and/or CO₂ each may be monitored and controlled by an optical patch and sensor mounted on, e.g., a gamma-irradiatable, biocompatible polymer which, can be sealed to, embedded in, or otherwise attached to the surface of the container.

As a specific example of a sensor, as shown in the embodiment illustrated in FIG. 5, container 18 may be operatively associated with a temperature controller 106 which may be, for example, a heat exchanger, a closed loop water jacket, an electric heating blanket, or a Peltier heater or cooler. Other heaters for heating a liquid inside a container are known to those of ordinary skill in the art and can also be used in combination with container 18. The heater may also include a thermocouple and/or a resistance temperature detector (RTD) for sensing a temperature of the contents inside the container. The thermocouple may be operatively connected to the temperature controller to control temperature of the contents in the container. Optionally, as described in more detail below, a thermally-conductive material may be associated with a surface of the container, e.g., to provide a heat transfer surface to overcome the insulating effect of the material used to form portions of the container.

Cooling may also be provided by a closed loop water jacket cooling system, a cooling system mounted on the reactor, or by standard heat exchange through a cover/jacket on the reusable support structure (e.g., the heat blanket or a packaged dual unit which provides heating and cooling may a component of a device configured for both heating/cooling but may also be separate from a cooling jacket). Cooling may also be provided by Peltier coolers. For example, a Peltier cooler may be applied to an exhaust line to condense gas in the exhaust air to help prevent an exhaust filter from wetting out.

In certain embodiments, a reactor system includes gas cooling for cooling the head space and/or exit exhaust. For example, jacket cooling, electrothermal and/or chemical cooling, or a heat exchanger may be provided at an exit air line and/or in the head space of the container. This cooling can enhance condensate return to the container, which can reduce exit air filter plugging and fouling. In some embodiments, purging of pre-cooled gas into the head space can lower the dew point and/or reduce water vapor burden of the exit air gas.

Although the above-mentioned methods can be used to heat or cool the contents inside a container, the rate of heat exchange using such methods may be less than desirable in certain instances. In some cases, the rate of heat exchange is limited below desirable or optimal levels by the material used to form the container. For instance, containers for mixing and/or for use in performing biological, chemical, and/or pharmaceutical reactions, especially systems involving the use of disposable liners in the form of collapsible bags, are generally made of low thermally-conductive materials such as polyethylene, polytetrafluoroethylene (PTFE), or ethylene vinyl acetate. If the chemical/biochemical/physical process performed in the container gives off heat and the heat should be removed, e.g., for the purposes of maintaining a suitable growth environment or controlling a reaction, the use of low thermally-conductive materials may inhibit or slow heat extraction from the container to an undesirable degree. For example, highly exothermic chemical reactions, if not controlled, can produce run away heat generation and produce undesired byproducts and/or create a dangerous safety condition of overpressure and/or over-temperature. Furthermore, in cases where a bioreaction is rapid and energetic, it may be desirable for heat to be removed to maintain the culture within the operating temperature range for optimum cell growth and/or product formation. In certain embodiments involving engineered organisms, product formation is controlled by heat-sensitive promoters that are activated by a rapid temperature shift. In these and other cases, heat removal rates from the container are important to control the amount of product formation. Cooling the harvested culture after the production run may also require rapid heat removal.

As containers are scaled up in size, the ratio of the surface area of the container to the liquid volume of the container is decreased. This reduces the amount of effective cooling capability of the container and can make temperature control for large containers more challenging. To address this problem, containers described herein, such as collapsible bags or rigid containers, include in certain embodiments one or more thermally-conductive material(s) associated therewith. In one embodiment, the container comprises a thermally-conductive material embedded in at least a portion of a wall of the container. Additionally or alternatively, the thermally-conductive material may line a wall of the container. For instance, the thermally-conductive material and the wall of the container may form a laminate structure. In vessels including one or more bladders, a thermally-conductive material may also be used to form all or portions of a bladder to facilitate heat transfer. Other configurations are also possible, as described in more detail below.

Advantageously, the container (and/or bladder) may be formed and configured such that the thermally-conductive material is adapted to conduct heat away from an interior of the container to an environment outside of the container, or to conduct heat into the container from an environment outside of the container. In embodiments in which the container is supported by a reusable support structure (e.g., a stainless steel tank), heat conduction away from or into the container can be facilitated by the support structure. For instance, heat from the contents inside the container can be dissipated, via the thermally-conductive material of the container, to the support structure which may also be thermally-conductive. The support structure may optionally be cooled using a suitable cooling system to enhance the rate of heat dissipation.

In some embodiments, the thermally-conductive material is in the form of a plurality of particles. The particles may be in the form of nanoparticles, microparticles, powders, and the like. The thermally-conductive material may also be in the form of nanotubes, nanowires, nanorods, fibers, meshes, or other entities. The thermally-conductive material can be embedded in the material used to form the container, e.g., such that all or a portion of each entity is enveloped or enclosed by the material used to form the container.

In some embodiments, an embedded thermally-conductive material is substantially uniformly dispersed throughout a bulk portion of a material used to form a container. “Substantially uniformly dispersed,” in this context, means that, upon viewing a cross-sectional portion of any such material, where the cross-section comprises the average makeup of a number of random cross-sectional positions of the material, investigation of the material at a size specificity, e.g., on the order of grains, or atoms, reveals essentially uniform dispersion of the thermally-conductive material in the bulk material. The number of random cross-sectional portions used to determine the average may be, for example, at least 3, at least 5, at least 10, or at least 20. In some cases, the number of random cross-sectional portions is chosen such that the addition of one more cross-sectional portion does not change the average by more than 5%, or in other embodiments, by no more than 1%. A photomicrograph, scanning electron micrograph, or other similar microscale or nanoscale investigative process may reveal essentially uniform distribution. “A bulk portion” of a material includes at least 50% of a cross-sectional dimension of the material. In certain embodiments, a bulk portion may comprise at least 60%, 70%, 80%, 90%, or 95% of a cross-sectional dimension of the material. Those of ordinary skill in the art, with this description, will understand clearly the meaning of these terms.

It should be understood that in other embodiments, a thermally-conductive material is not substantially uniformly dispersed throughout a bulk portion of the material used to form a container (and/or bladder). For example, a gradient of particles may be formed across a cross-section of the container. In another example, a thermally-conductive material may form a film or layer adjacent a layer of a material used to form the container. In some such embodiments, the film or layer of thermally-conductive material is uniformly positioned across a width or height of the container. For example, the thermally-conductive material may be configured such that one portion of the container includes a thermally-conducive material and another, adjacent portion of the container also comprises the thermally-conductive material. Alternatively, the thermally-conductive material may be present as strips, wires, or may have other configurations such that one portion of the container includes a thermally-conducive material and another, adjacent portion of the container does not comprise a thermally-conductive material.

The thermally-conductive material may in certain embodiments be encapsulated between two polymeric sheets. Alternating layers of thermally-conductive material and polymeric layers are also possible. Alternatively, in some embodiments, an outer surface of the container may include a layer of thermally-conductive material, while an inner surface of the container does not include the thermally-conductive material. This configuration may allow heat to be conducted away from (or into) the contents of the container, while avoiding or limiting any reactivity between the contents of the container and the thermally-conductive material. For example, silver has a high thermal conductivity and may be used as a thermally-conductive material, but is known to have antimicrobial effects. By positioning the silver at an outer surface of the container (or embedded between two polymer layers), but not in contact with any contents inside the container, heat conduction of the container may be enhanced without adversely affecting the contents inside the container (e.g., cells, proteins, etc.).

The thermally-conductive material may have any suitable size or dimension. The size of the thermally-conductive entities may be chosen, for example, to achieve a certain dispersion (e.g., a gradient or a substantially uniformly dispersion) within the bulk material used to form the container, to prevent protrusion of the entity through a portion of the container, or to have a certain surface area or thermally conductive material to container volume ratio. In some cases, the thermally-conductive material has at least one cross-sectional dimension less than 500 microns, less than 250 microns, less than 100 microns, less than 50 microns, less than 10 microns, less than 1 micron, less than 100 nanometers, less than 50 nanometers, less than 25 nanometers, less than 10 nanometers, less than 5 nanometers, or less than 1 nanometer.

Any suitable material thermally conducting material can be used as a thermally-conductive material. The thermally-conductive material may be chosen based on factors such as its thermal conductivity, particle size, magnetic properties, compatibility with certain processing techniques (e.g., ability to be deposited by certain deposition techniques), compatibility with the bulk material used to form the container, compatibility with any materials contained in the container (e.g., cells, nutrients, gases, etc.), compatibility with any treatments or pre-treatments associated with performing a reaction inside the container (e.g., sterilization), as well as other factors.

In one specific set of embodiments, the thermally-conductive material comprises a metal. In one embodiment, the thermally-conductive material is a metal. In other cases, the thermally-conductive material comprises a semiconductor. Materials potentially suitable for use as thermally-conductive materials include, for example, a Group 1-17 element, e.g., specifically, a Group 2-14 element, or a Group 2, 10, 11, 12, 13, 14, 15 element. Potentially suitable elements from Group 2 of the Periodic Table may include beryllium, magnesium, calcium, strontium, and barium. Potentially suitable elements from Group 10 may include, for example, nickel, palladium, or platinum. Potentially suitable elements from Group 11 may include, for example, copper, silver, or gold. Potentially suitable elements from Group 12 may include, for example, zinc, cadmium, or mercury. Elements from Group 13 that may be suitable include, for example, boron, aluminum, gallium, indium, or thallium. Elements from Group 14 that may be suitable include, for example, carbon, silicon, germanium, tin, or lead. Elements from Group 15 that may be suitable include, for example, nitrogen, phosphorus, or bismuth. In some cases, the thermally-conductive material is Al, Cu, Fe, or Sn.

Where the thermally-conductive material comprises a metal, it is to be understood that one or more metals can be used. Similarly, where the thermally-conductive material comprises a semiconductor, one or more semiconducting materials can be used. Additionally, metals and semiconductors can be mixed. That is, the thermally-conductive material can be a single metal, a single semiconductor, or one or more metals or one or more semiconductors mixed (e.g., an alloy). Non-limiting examples of suitable metals are listed above, and suitable components of semiconductors are listed above. Those of ordinary skill in the art are well aware of semiconductors that can be formed from one or more of the elements listed above, or other elements.

In certain cases, the thermally-conductive material is a nonmetal. For example, the thermally-conductive material may comprise carbon. The thermally-conductive material may be in the form of a conductive polymer, for instance. Non-limiting examples of conductive polymers include polypyrroles, polyanilines, polyphenylenes, polythiophenes, and polyacetylenes.

Those of ordinary skill in the art can easily select, from materials described above or other materials known in the field, suitable metals, semiconductors, and/or nonmetals. In addition, given the teachings described herein, one of ordinary skill in the art can screen materials for suitable use in connection with embodiments described herein without undue burden or undue experimentation.

Optionally, thermally-conductive materials may be coated or treated, e.g., chemically and/or physically, to enhance certain chemical and/or physical properties of the materials. For instance, the surfaces of the thermally-conductive materials may be treated, e.g., with a surfactant, an oxide or any other suitable material, to make the materials more hydrophilic/hydrophobic, less reactive, have a certain pH, etc. These and other processes can allow the thermally-conductive materials to be more compatible with the material used to form the container and/or with certain processing techniques. For example, treatment of the thermally-conductive material may allow it to adhere to the material used to form the container to a desired degree, be more soluble in a particular solvent, or achieve a certain level of dispersibility.

As described herein, in some embodiments, a container (e.g., a rigid container or a collapsible bag) and/or a bladder comprises a polymeric material (e.g., as a bulk material). Polymeric materials, such as the ones described herein, can be selected or formulated to have suitable physical/mechanical characteristics, for example, by tailoring the amounts of components of polymer blends, adjusting the degree of cross-linking (if any), etc. For instance, those of ordinary skill in the art can choose suitable polymers for use in containers based on factors such as the polymer's thermal conductivity, compatibility with certain processing techniques (e.g., ability to be deposited by certain deposition techniques), compatibility with thermally-conductive materials, compatibility with any materials contained in the container (e.g., cells, nutrients, gases, etc.), and compatibility with any treatments or pre-treatments associated with performing a reaction inside the container (e.g., sterilization).

Containers and/or bladders comprising a thermally-conductive material may be formed by any suitable method. In one embodiment, a thermally-conductive material is physically mixed with a material used to form the container (e.g., a bulk material), optionally with other components such as reactants, solvents, gases, and surfactants. The thermally-conductive material may be injected into the bulk material, for example. The resulting mixture may be in the form of a solution, emulsion, or suspension.

The mixture may be shaped into a container (or bladder), or a precursor of a container (or bladder), by a method such as blow molding, injection molding, spin cast molding, and extrusion, for instance, as described above and/or by methods known to those of ordinary skill in the art. For example, in one embodiment, the thermally-conductive material and material used to form the container may be co-extruded at a sufficiently high temperature at which the materials are pliable. The materials can then be shaped into a container or a precursor to the container such as a sheet. Containers including thermally-conductive materials may be seamless or may include seams that are welded together to form the container. In some cases, more controlled welding can be achieved by heating the thermally-conductive material by an energy source such as a microwave source or a laser.

In some embodiments, the thermally-conductive material is applied to all or a portion of a material used to form the container or bladder by methods such as physical deposition methods, chemical vapor deposition methods, plasma enhanced chemical vapor deposition techniques, thermal evaporation (e.g., resistive, inductive, radiation, and electron beam heating), sputtering (e.g., diode, DC magnetron, RF, RF magnetron, pulsed, dual magnetron, AC, FM, and reactive sputtering), jet vapor deposition, electrophoretic deposition, magnetophorectic deposition, spin coating, dip coating, spraying, brushing, screen printing, ink-jet printing, toner printing, sintering, laser ablation, electroplating, ion plating, cathodic arc, and combinations thereof. Such methods can be carried out in a vacuum or inert atmosphere.

The thermally-conductive material may optionally be aligned, especially in embodiments in which the materials are embedded in a bulk material, using magnetic interactions, electrostatic interactions, and the like.

The container (or bladder or any other article) may include any suitable amount of thermally-conductive material. The container may comprise, for example, at least 0.1 wt %, at least 1 wt %, at least 2 wt %, at least 5 wt %, at least 10 wt %, at least 15 wt %, at least 20 wt %, at least 30 wt %, or at least 50 wt % of thermally-conductive material, e.g., based on the total weight of the container. In some cases, these percentages are based on the total weight of the flexible portions (e.g., the wall portions) of the container.

The amount and type of thermally-conductive material(s), the material used to form the container, the arrangement of the thermally-conductive material with respect to the material used to form the container, and the thickness of the container can be chosen such that the container achieves a certain overall level of thermal conductivity. The overall thermal conductivity of the container may be, for example, at least 0.1 Watts·m⁻¹ K⁻¹, at least 0.2 Watts·m⁻¹·K⁻¹, at least 0.5 Watts·m⁻¹·K⁻, at least 1 Watts·m⁻¹·K⁻, at least 2 Watts·m⁻·K⁻¹, at least 3 Watts·m⁻¹·K⁻¹, at least 5 Watts·m⁻¹·K⁻¹, at least 10 Watts·m⁻¹·K⁻¹, or at least 15 Watts·m⁻¹·K⁻¹. In some cases, the thermal conductivity of a container including a thermally-conductive material is at least 1.5 times, at least 2 times, at least 5 times, at least 10 times, or at least 50 times greater than a container without a thermally-conductive material (all other factors being equal). The thermal conductivity can be measured by those of ordinary skill in the art by determining the quantity of heat transmitted, during a period of time, through a thickness of the container in a direction normal to a surface area of the container, wherein the quantity of heat transmitted is due to a temperature difference under steady state conditions and when the heat transfer is dependent only on the temperature gradient.

In addition to the benefits of enhanced heat conduction using containers and/or bladders comprising thermally-conductive materials, such articles may also have enhanced sensing capabilities. For instance, the containers may be use to determine temperature, conductance, impedance, as well as dissipation and control of static charge. In some embodiments, the containers can be used to detect any leakage of materials from the interior to the outside of the container. Such measurements can be performed, for example, by determining a change in the thermal and/or electrical conductivity of one or more portions of the container.

Referring now to FIG. 5, in some cases, sensors and/or probes (e.g., probe 106) may be connected to a sensor electronics module 132, the output of which can be sent to a terminal board 130 and/or a relay box 128. The results of the sensing operations may be input into a computer-implemented control system 115 (e.g., a computer) for calculation and control of various parameters (e.g., temperature and weight/volume measurements) and for display and user interface. Such a control system may also include a combination of electronic, mechanical, and/or pneumatic systems to control heat, air, and/or liquid delivered to or withdrawn from the disposable container as required to stabilize or control the environmental parameters of the process operation. It should be appreciated that the control system may perform other functions and is not limited to having any particular function or set of functions.

The one or more control systems can be implemented in numerous ways, such as with dedicated hardware and/or firmware, using a processor that is programmed using microcode or software to perform the functions recited above or any suitable combination of the foregoing. A control system may control one or more operations of a single reactor for a biological, biochemical or chemical reaction, or of multiple (separate or interconnected) reactors.

Each of systems described herein (e.g., with reference to FIG. 5), and components thereof, may be implemented using any of a variety of technologies, including software (e.g., C, C#, C++, Java, or a combination thereof), hardware (e.g., one or more application-specific integrated circuits), firmware (e.g., electrically-programmed memory) or any combination thereof.

Various embodiments described herein may be implemented on one or more computer systems. These computer systems, may be, for example, general-purpose computers such as those based on Intel PENTIUM-type and XScale-type processors, Motorola PowerPC, Motorola DragonBall, IBM HPC, Sun UltraSPARC, Hewlett-Packard PA-RISC processors, any of a variety of processors available from Advanced Micro Devices (AMD) or any other type of processor. It should be appreciated that one or more of any type of computer system may be used to implement various embodiments described herein. The computer system may include specially-programmed, special-purpose hardware, for example, an application-specific integrated circuit (ASIC). Various components may be implemented in software, hardware or firmware, or any combination thereof. Further, such methods, acts, systems, system elements and components thereof may be implemented as part of the computer system described above or as an independent component.

In one embodiment, a control system operatively associated with a vessel described herein is portable. The control system may include, for example, all or many of the necessary controls and functions required to perform a fluidic manipulation (e.g., mixing and reactions) in the control system. The control system may include a support and wheels for facilitating transport of the vessel. Advantageously, such a portable control system can be programmed with set instructions, if desired, transported (optionally with the vessel), and hooked up to a vessel, ready to perform a fluid manipulation in a shorter amount of time than conventional fluid manipulation control systems (e.g., less than 1 week, 3 days, 1 day, 12 hours, 6 hours, 3 hours, or even less than 1 hour).

A vessel may also be connected to one or more sources of gases such as air, oxygen, carbon dioxide, nitrogen, ammonia, or mixtures thereof, in some embodiments. The gases may be compressed, pumped, etc. Such gases may be used to provide suitable growth and/or reaction conditions for producing a product inside the container. The gases may also be used to provide sparging to the contents inside the container, e.g., for mixing or other purposes. For instance, in certain embodiments employing spargers, bubble size and distribution can be controlled by passing an inlet gas stream through a porous surface prior to being added to the container. Additionally, the sparging surface may be used as a cell separation device by alternating pressurization and depressurization (or application of vacuum) on the exterior surface of the porous surface, or by any other suitable method.

As a specific example, FIG. 5 shows various sources of gases 118 and 124. The inlet gases may optionally pass through filter 120 and/or a flow meter and/or valve 122, which may be controlled by controller system 115, prior to entering the container. Valve 122 may be a pneumatic actuator (actuated by, e.g., compressed air/carbon dioxide or other gas 124), which may be controlled by a solenoid valve 126. These solenoid valves may be controlled by a relay 128 connected to terminal board 130, which is connected to the controller system 115. The terminal board may comprise, for example, a PCI terminal board, or a USB/parallel, or fire port terminal board of connection. In other embodiments, flush closing valves can be used for addition ports, harvest and sampling valves. Progressive tubing pinch valves that are able to meter flow accurately can also be used. In some cases, the valves may be flush closing valves (e.g., for inlet ports, outlet ports, sampling ports, etc.). The inlet gases may be connected to any suitable inlet of the vessel. In one embodiment, the inlet gases are associated with one or more spargers which can be controlled independently, as described in more detail below.

As shown in the exemplary embodiment illustrated in FIG. 5, the container and support structure illustrated in FIG. 1 can be operatively associated with a variety of components as part of an overall bioreactor system 100. Accordingly, the container and/or support structure may include several fittings to facilitate connection to functional component such as filters, sensors, and mixers, as well as connections to lines for providing reagents such as liquid media, gases, and the like. The container and the fittings may be sterilized prior to use so as to provide a “sterile envelope” protecting the contents inside the container from airborne contaminants outside. In some embodiments, the contents inside the container do not contact the reusable support structure and, therefore, the reusable support structure can be reused after carrying out a particular chemical, biochemical and/or biological reaction without being sterilized, while the container and/or fittings connected to the container can be discarded. In other embodiments, the container, fittings, and/or reusable support structure may be reused (e.g., after cleaning and sterilization).

A vessel may also include a mixing system for mixing contents of the container, in another aspect. In some cases, more than one agitator or mixer may be used, and the agitators and/or mixes may the same or different. More than one agitation system may be used, for example, to increase mixing power. In some cases, the agitator may be one in which the height can be adjusted, e.g., such that the draft shaft allows raising of an impeller or agitator above the bottom of the tank and/or allows for multiple impellers or agitators to be used. A mixing system of a vessel may be disposable or intended for a single use (e.g., along with the container), in some cases.

Various methods for mixing fluids can be implemented in the container. For instance, mixers based on magnetic actuation, sparging, and/or air-lift can be used. Direct shaft-drive mixers that are sealed and not magnetically coupled can also be used. In one particular embodiment, mixing systems such as the ones disclosed in U.S. patent application Ser. No. 11/147,124, filed Jun. 6, 2005, entitled “Disposable Bioreactor Systems and Methods,” by G. Hodge, et al., published as U.S. Patent Application Publication No. 2005/0272146 on Dec. 8, 2005, which is incorporated herein by reference in its entirety, are used with embodiments described herein. For example, the mixing system may include a motor 112, e.g., for driving an impeller (or other component used for mixing) positioned inside the container, a power conditioner 114, and/or a motor controller 116.

In some cases, a plurality (e.g., more than 1, 2, or 3, etc.) of mixers or impellers are used for mixing contents in a container. Additionally and/or alternatively, a mixing system may include an adjustable height impeller and/or an impeller with varying impeller blade configurations. For instance, the mixer may have an extended drive shaft which allows the impeller to be raised to different heights relative to the bottom of the container. The extended shaft can also allow integration of multiple impellers. In another embodiment, a bioreactor system includes more than one agitation drive per container, which can increase mixing power.

To enhance mixing efficiency, the container may include baffles such as internal film webs or protrusions, e.g., positioned across the inside of the container or extending from the inner surface of the container at different heights and at various angles. The baffles may be formed of in any suitable material such as a polymer, a metal, or a ceramic so long as they can be integrated with the container.

In one embodiment, a direct drive agitator is used. Typically, the agitator includes a direct shaft drive that is inserted into the container. In certain instances, the location where the shaft exits the container may be maintained in a sterile condition. For instance, internal and/or external rotating seals may be used to maintain a sterile seal, and/or live hot steam may be used to facilitate maintenance of the sterile seal. By maintaining such a sterile seal, contamination caused by the shaft, e.g., from the external environment, from the exiting gases, etc., may be reduced or avoided.

In certain embodiments, a magnetic agitator is used. A magnetic agitator may use magnets such as fixed, permanent, or electromagnets to rotate or otherwise move the agitator, for example, impellers, blades, vanes, plates, cones, etc. In some cases, the magnets within the magnetic agitator are stationary and can be turned on or activated in sequence to accelerate or decelerate the agitator, e.g., via an inner magnetic impeller hub. As there is no penetration of the container by a shaft, there may be no need to maintain the agitator in a sterile condition, e.g., using internal and/or external rotating seals, live hot steam, or the like.

In yet another embodiment, an electromechanical polymeric agitator is used, e.g., an agitator that includes an electromechanical polymer-based impeller that spins itself by “paddling,” i.e., where the agitator is mechanically flapped to propel the agitator or impeller, e.g., rotationally.

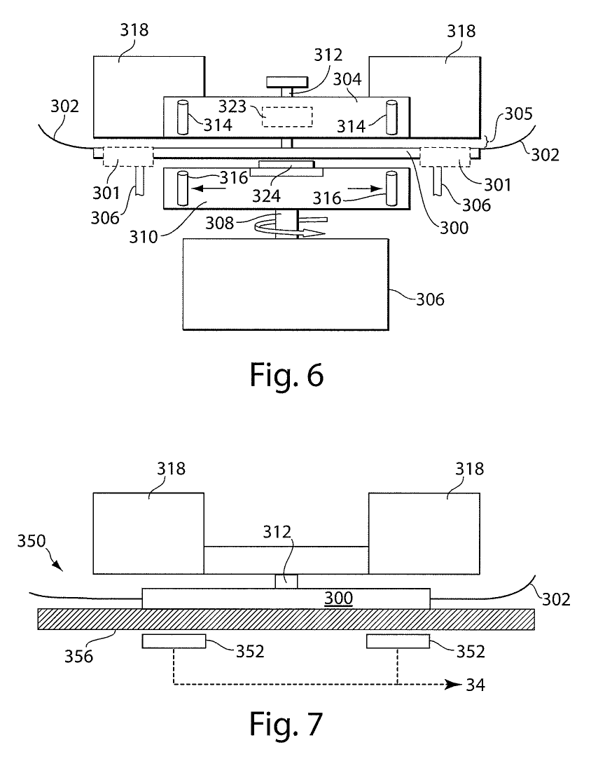

Specific non-limiting examples of devices that can be used as a mixing system, and/or an antifoaming system in certain embodiments, are illustrated in FIGS. 6-9. The devices shown include a magnetically-actuated impeller, although other arrangements are possible. In some of these magnetic configurations, the motor is not directly connected to the impeller. Magnets associated with a drive head can be aligned with magnets associated with an impeller hub, such that the drive head can rotate the impeller through magnetic interactions. In some cases, the motor portion (and other motor associated components) may be mounted on the support structure.

As shown in FIG. 6, this exemplary system generally includes an impeller support 300 affixed to portions of a container wall 302, preferably at a lower portion thereof, an impeller hub 304, a motor 306, a motor shaft 308 and a drive head 310. The impeller support may be affixed to the wall of the container using any suitable technique, e.g., by heat welding together two portions of a two-piece impeller support, sandwiching the container wall therebetween or onto the wall, or using other methods described herein. As one example, an opening in the wall of the container may be used to allow a central portion of the impeller plate to extend from an exterior of the container to the interior (or vise versa). Then a sealing ring (not shown) may be adhered or the container may be welded directly to an outer circumference of the impeller support to seal the container wall therebetween. As another example, an undersized opening in the wall of the container may be used to form a seal with a circumferencial edge of the impeller support slightly larger than the opening. In other embodiments, at least a portion of the impeller support is embedded with a wall of the container and/or the impeller support and container are fabricated simultaneously (e.g., by spin casting, injection molding, or blow molding).

One feature of some embodiments described herein is directed to the inclusion of one or more spargers associated with an impeller support, which may be used to direct air or other gases into the container. In some cases, the sparger may include porous, micro-porous, or ultrafiltration elements 301 (e.g., sparging elements). The spargers may be used to allow a gaseous sparge or fluids into and/or out of the container by being dimensioned for connection to a source of a gas; this connection may take place via tubing 306. Such sparging and/or fluid addition or removal may be used, in some cases, in conjunction with a mixing system (e.g., the rotation of the impeller hub). Sparging systems are described in more detail below.

In the embodiment illustrated in FIG. 6, the interior side of the impeller support may include a shaft or post 312 to which a central opening in the impeller hub 304 receives. The impeller hub may be maintained at a slight distance 305 above the surface of the impeller support (e.g., using a physical spacer) to prevent friction therebetween. Low friction materials may be used in the manufacture of the impeller hub to minimize friction between the impeller hub and the post. In another embodiment, one or more bearings may be included to reduce friction. For instance, the impeller hub may include, in certain instances, a bearing 323 (e.g., a roller bearing, ball bearing (e.g., a radial axis ball bearing)), thrust bearing, race bearing, double raceway bearing, lazy-susan bearing, or any other suitable bearing) for reducing or preventing friction between the impeller support and the post. Additionally, the drive head may include a physical spacer 324 for reducing or preventing friction between the drive head and the impeller support.

The impeller hub also may include one or more impeller blades 318. In some cases, the embedded magnet(s) in the impeller can also be used to remove ferrous or magnetic particles from solutions, slurries, or powders.

The impeller hub also may include one or more magnets 314, which may be positioned at a periphery of the hub or any other suitable position, and may correspond to a position of a magnet(s) 316 provided on the drive head 310. The poles of the magnets may be aligned in a manner that increases the amount of magnetic attraction between the magnets of the impeller hub and those of the drive head. Magnets 314 and/or 316 may be permanent magnets, electromagnets, superconducting magnets, or combinations thereof. For instance, in one embodiment, magnets 314 are permanent magnets and magnets 316 are electromagnets. In another embodiment, magnets 314 are electromagnets and magnets 316 are permanent magnets. In some cases, the system comprises a single or series of electromagnets sequenced either manually or electronically (e.g., solid state relays). Other combinations are also possible.

The drive head 310 may be centrally mounted on a shaft 308 of motor 306. In mixing systems including electromagnets, drive head 310 may be powered by a signal generator. The signal generator can be programmed to operate the electromagnet(s) with a particular frequency and/or current to regulate the strength of the magnetic field, and therefore, the strength of the coupling interaction with the impeller and the degree of mixing in the container.

It should be understood that not all of the features shown in FIG. 6 need be present in all embodiments of the invention and that the illustrated elements may be otherwise positioned or configured. For instance, in some embodiments involving electromagnets, shaft 308 and/or motor 306 may not be required as the electromagnets can be operated sequentially to impart a rotating magnetic force, e.g., using a signal generator. Also, additional elements may be present in other embodiments, such as the elements described herein.

Advantageously, mixing systems that include electromagnets can allow the magnetic attraction between the impeller and the drive head to be controlled. For instance, the electromagnets can be turned off to afford easy insertion and/or removal of the mixing system components, such as removal of the drive head from impeller support 300.

Another example of an electromagnetically-driven impeller is shown in the embodiment illustrated in FIG. 7. Mixing system 350 includes impellers 318 that can rotate about a post or shaft 312. The post or shaft can be connected to impeller support 300, which may be affixed to portions of a container wall 302. The impeller support and/or container wall may be contained in and/or supported by a wall 356 of a reusable support structure. As illustrated, wall 356 of the support structure does not include an opening that exposes impeller support 300. This arrangement can prevent any fluids that may leak out of the container from leaking out of the support structure. This configuration can also reduce or eliminate problems with friction associated with a rotating hub. In other embodiments, however, an opening in the wall of the support structure may be present, which can allow direct access to impeller support 300.

Electromagnets 352 may be positioned exterior to the wall of the support structure. In one embodiment, the electromagnets are positioned in the form of a circular ring and can be operated sequentially to impart a rotating magnetic force to the impellers. The electromagnets may be in electrical communication with a control system 34 which may include a signal generator and/or other controls for operating the electromagnets.

In some embodiments, none of the electromagnetic force is diverted to suspend the impeller. That is, substantially all of the magnetic force generated can be used to impart rotational motion since the impeller may be mounted on a fixed bearing, and does not have to be suspended. This feature can prevent the impeller from accidently contacting the container (e.g., collapsible bag) and damaging it.

Additional examples of mixing systems and components that can be used in such systems are described in U.S. patent application Ser. No. 11/147,124, filed Jun. 6, 2005, entitled “Disposable Bioreactor Systems and Methods,” by G. Hodge, et al., published as U.S. Patent Application Publication No. 2005/0272146 on Dec. 8, 2005, and in U.S. Patent Application Publication No. 20020118594, filed Feb. 27, 2002 and entitled, “Apparatus and method for mixing small volumes of liquid,” which are incorporated herein by reference.

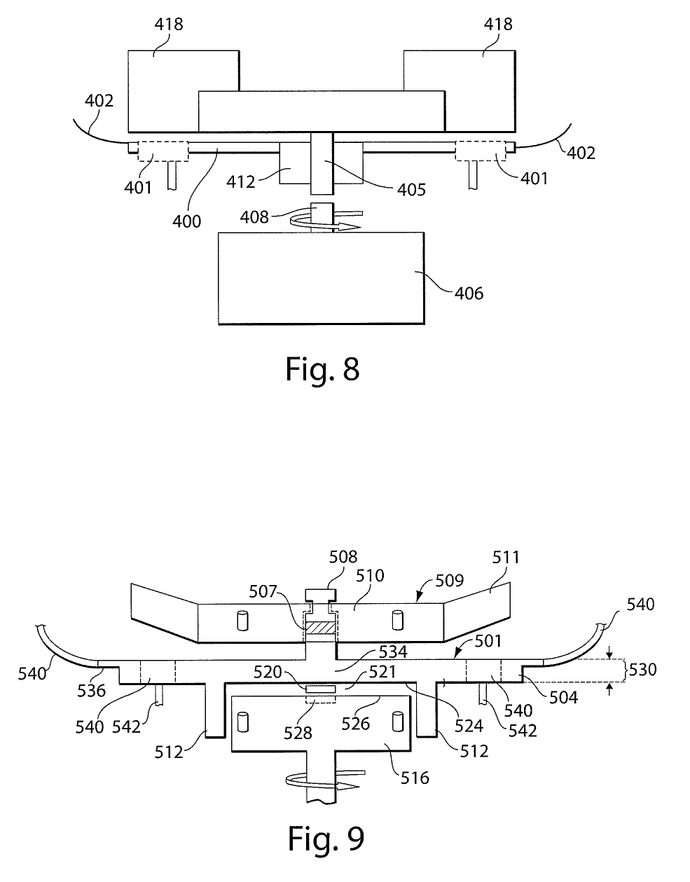

FIG. 8 illustrates another embodiment, having a mechanically-driven impeller. As shown, this embodiment generally includes an impeller support 400, an impeller hub 404 with shaft 405, and an external motor 406 with shaft 408. The connection of shafts between the impeller hub shaft and the motor shaft may be accomplished in a matter familiar to one of ordinary skill in the art (e.g., gear box, hex drive, or the like).

The impeller support can be affixed, for instance, to a side of the bioreactor wall 402 at a lower portion thereof. The impeller support may be affixed to the wall of the bioreactor by any of the methods discussed herein. Porous, micro-porous, or ultrafiltration elements 401 may also be included in the present embodiment to allow gaseous sparge or fluids into and out of the bioreactor, as discussed in detail below. In the embodiment illustrated in FIG. 8, the shaft of the impeller hub may be received in a seal 412 (which may also include a bearing, in some cases) centrally located in an impeller support 400. The seal can be used to insure that the contents of the container are not contaminated. The impeller hub can also be maintained at a slight distance above the surface of the impeller support to prevent friction therebetween. The impeller hub may include one or more impeller blades 418, or other suitable mixing structures, such as vanes, plates, cones, etc.

Referring now to FIG. 9, one embodiment of a drive head magnetically coupled to an impeller is illustrated schematically. In FIG. 9, an impeller support 501, shown in a cross-section, includes a substantially horizontal portion 504, from which a substantially vertical impeller shaft 508 extends upwardly supporting an impeller 509 (which may include a core 510 and blades 511). Impeller 509 may rotate about shaft 508. Optionally, this rotation may be facilitated by a bearing 507, which may be any suitable bearing such as a roller bearing, ball bearing (e.g., a radial axis ball bearing), thrust bearing, race bearing, double raceway bearing, lazy-susan bearing, or the like.

Impeller support 501 includes drive head alignment elements 512 which, in the embodiment illustrated, are substantially vertical downwardly-depending ridges which can define a circular recess into which at least a portion of a drive head 516 can be inserted. Guide elements 512 are positioned such that drive head, when engaged with the impeller support, position the drive head at a predetermined desired location relative impeller 509. In one arrangement, guide elements 512 center the drive head, when engaged with the impeller support, with respect to impeller 509.

As a further, optional embodiment, a physical spacer 520 can be provided between drive head 516 and a bottom surface 524 of the impeller support aligned with that portion of the top surface 526 of the drive head at the location at which the drive head is ideally positioned with respect to the impeller support. Physical spacer 520 physically separates, by a desired distance, the bottom surface 524 of the impeller support with a top surface 526 of the drive head, but, at least one portion between the top surface of the drive head and bottom surface of the impeller support, may define a continuous, physical connection (free of voids of air or the like), between the drive head and the impeller support. This allows for closer tolerance of the drive head with the impeller support than would have been realized in many prior arrangements, and it allows for reproducible and secure engagement of the drive head with the impeller support. In some cases, the drive head includes a recess 528 into which at least a portion of physical spacer 520 can be inserted. This arrangement can allow reproducible and secure engagement of the drive head with the physical spacer.

The bottom of the impeller support and the top surface of the drive head can be separated (e.g., using a physical spacer) by a distance 521. In one embodiment, distance 521 is no greater than 50% of average thickness 530 of the substantially horizontal portion 504 of the impeller support. In other embodiments, this distance is no more than 40%, 30%, 20%, 10%, or 5% of the thickness of the impeller support.

In some embodiments, physical spacer 520 has a thickness no greater than 50% of average thickness 530 of the substantially horizontal portion 504 of the impeller support. In other embodiments, this thickness is no more than 40%, 30%, 20%, 10%, or 5% of the thickness of the impeller support.

In one set of embodiments, physical spacer 520 is a bearing that facilitates rotation of the drive head relative to the impeller support. Where physical spacer 520 is a bearing, any suitable bearing can be selected such as a roller bearing, ball bearing (e.g., a radial axis ball bearing), thrust bearing, race bearing, double raceway bearing, lazy-susan bearing, or the like.

In the embodiment illustrated in FIG. 9, the drive head can vary in position, relative to shaft 508, horizontally no more than 5 mm during normal operation or, in other embodiments, no more than 4, 3, 2, 1 (0.5, or 0.25 mm during normal operation). The drive head can also vary in distance relative to bottom surface 524 of the impeller support by no more than 10 mm, 1 mm, 0.5 mm, 0.25 mm, 0.1 mm, or 0.005 mm in certain embodiments with the use of the arrangements illustrated in FIG. 9.

The arrangements of FIG. 9, especially in embodiments where physical spacer 520 is used, also adds physical support to impeller support 501 in addition to any other physical support which the impeller support 501 might receive. This added support is particularly advantageous in collapsible bag arrangements including impellers (e.g., for mixers and/or antifoaming devices).

The impeller hub and drive head may include one or more magnets 314 and 316, which may include fixed or permanent magnets, electromagnets, superconducting magnets, or combinations thereof, as described above. Although the magnets are shown to be positioned at a periphery of the hub, it should be understood that magnets 314 and 316 may have any suitable size and configuration, and may be positioned at any suitable location with respect to the impeller and the drive head. As mentioned above, the use of electromagnets can allow the magnetic attraction between the impeller and the drive head to be controlled, which can facilitate insertion and/or removal of drive head 516 from the recess formed by drive head alignment elements 512.

Optionally, impeller support 501 may include spargers 540 positioned beneath blades of the impeller. The spargers can be dimensioned for connection to one or more sources of gas. For example, the spargers may include a port that can be connected to tubing 542 in fluid communication with one or more sources of gas.

Although many of the figures described herein show impellers that are positioned at or near a bottom portion of a container, in other embodiments, impellers can be positioned at any suitable location within a container, for example, near the center or a top portion of a container. This can be achieved by extending the length of a shaft which supports the impeller, or by any other suitable configuration. Positions of impellers in a container may depend on the process to be performed in the container. For instance, in some embodiments where sparging is required, impellers may be positioned near the sparger such that the impeller can sweep and/or regulate the bubbles introduced into the container. Additionally, although the figures described herein show a single impeller associated with a shaft, more than one impeller can be used in some instances. For example, a first impeller coupled to a shaft may be located near a bottom portion of the container and a second impeller coupled to the shaft may be positioned near the center of the container. The first impeller may provide adequate sweeping of a sparged gas, and the second impeller may provide adequate mixing of contents within the container.

In one embodiment, the impeller support is uniquely designed to be readily fastenable to a collapsible bag. Certain know arrangements of impellers attached to collapsible bags may suffer from drawbacks resulting from non-ideal attachment of the bag to the impeller support, or non-ideal techniques for such attachment, or both. As shown in the embodiment illustrated in FIG. 9, one embodiment may include an impeller support having a base, substantially perpendicular to a shaft upon which the impeller rotates, having a first portion 534 of average thickness sufficient to adequately support the impeller shaft, and a second, peripheral portion 536 thinner than the first portion for facilitating attachment to the bag. The first portion thickness is defined as the overall thickness cross-section taken up by the first portion at any point and, where the first portion includes a ribbed or other structure including various thicknesses, the thickness for purposes of this discussion is defined as the thickest portion. The second, peripheral portion, in one embodiment, defines a composition similar to or essentially identical to that of the collapsible bag, and is provided in a thickness similar to that of the collapsible bag. In other embodiments, the second, peripheral portion is formed by a composition different than that of the collapsible bag. For instance, in some embodiments, the first portion is formed in low density polyethylene, and the second portion is formed in high density polyethylene, polypropylene, silicone, polycarbonate, and/or polymethacrylate.

The thickness of the peripheral portion of the support and the thickness of the walls of collapsible bag 540, prior to attachment, may differ by no more than 100%, or by no more than 80%, 60%, 40%, 20%, or 10% in other embodiments (e.g., as a percentage of the greater thickness between the walls of the bag and the peripheral portion). Where the thickness of the peripheral portion of the impeller support and the thickness of the disposable bag (at least the portion attachable to the impeller support) are made of similar (or compatible) materials and are of similar thickness, then joining of one to the other may be facilitated easily, reproducibly, and with a product that is free of significant irregularity and thickness in the transition of the bag to the impeller support attachment portion. Thus, one embodiment may involve the product of attachment of a collapsible bag and an impeller support each as defined above, and in another aspect involves a kit including an impeller support and a collapsible bag prior to attachment. As described herein, joining of the bag and the support can be performed by any suitable method including, for example, molding and welding (e.g., ultrasonic or heat welding). In one aspect, the impeller (in some embodiments, via magnetic coupling of the drive head to the impeller) is driven by a motor able to reverse its direction of rotation and/or to be finely tuned with respect to rotational speed. Reversal of direction of spin provides significant advantage in terms of achieving a variety of aeration/sparger profiles. Fine tuning of impeller speed has been determined to allow for a precise and controllable degree and/or balance of aeration/sparging, sheer, or the like, which has been determined to be quite useful in connection with various media for mixture, especially those including cells. This embodiment allows for reproducible and controllable adjustment of rotational speed of the impeller that amounts of plus or minus 5% or less through a range of rotational speeds of between 10% and 90% of total maximum impeller rotational speed. In other embodiments, rotational tuning of 4%, 3%, 2%, or 1% of this speed is facilitated. In one arrangement, these aspects are realized by use of a servo motor.

The impeller systems described herein may allow the system to mix fluids, solids, or foams of any type. For example, fluids inside the container may be mixed to provide distribution of nutrients and dissolved gases for cell growth applications. The same disposable container may be used for mixing buffers and media or other solutions in which a disposable product contact surface is desirable. This may also include applications in which the vessel is not required to be sterile or maintain sterility. Moreover, embodiments described herein enable the container holding the fluids/mixtures/gases to be removed and discarded from the reusable support structure such that the reusable support structure is not soiled by the fluids that are mixed in the container. Thus, the reusable support structure need not to be cleaned or sterilized after every use.

In some embodiments, multiple spargers (including sparging elements) may be dimensioned for connection to different sources of gas and/or which may be independently controlled. The type of gas, number of spargers, and types and configurations of spargers used in a bioreactor system or a biochemical/chemical reaction system may depend, in part, on the particular process to be carried out (e.g., an aerobic versus anaerobic reaction), the removal of any toxic byproducts from the liquid, the control of pH of a reaction, etc. As described in more detail in U.S. patent application Ser. No. 11/818,901, filed Jun. 15, 2007, entitled, “Gas Delivery Configurations, Foam Control Systems, and Bag Molding Methods and Articles for Collapsible Bag Vessels and Bioreactors”, which is incorporated herein by reference, a system may include separate spargers for different gases which may have different functions in carrying out, for example, a chemical, biochemical and/or biological reaction. For instance, a bioreactor system for cell cultivation may include different types of gases such as a “dissolved oxygen (DO) control gas” for controlling the amount of dissolved oxygen in the culture fluid, a “strip gas” for controlling the amount of toxic byproducts in the culture fluid, and a “pH control gas” for controlling the pH of the culture fluid. Each type of gas may be introduced into the culture using different spargers that can be independently operated and controlled. Advantageously, such a system may provide faster process control and less process control variability (compared to, for example, certain systems that combine a DO control gas, strip gas, and pH control gas into one gas stream introduced into a reactor). Chemical, biochemical and/or biological reactions carried out in bioreactor systems described herein may also require lower consumption of gas which can save money on expensive gases, and/or less total gas flow rate (e.g., for a strip gas), which can reduce foam generation and/or reduce the size of inlet gas sterile filters required.

In one particular embodiment, a vessel (e.g., as part of a reactor system for performing a biological, biochemical or chemical reaction) is configured to contain a volume of liquid and includes a container (e.g., a collapsible bag) having a volume of at least 0.01 liters, or at least 2 liters (or any other suitable volume) to contain the volume of the liquid. The vessel may optionally include a support structure for surrounding and containing the container. Additionally, the vessel includes a first sparger connected or dimensioned to be connected to a source of a first gas composition in fluid communication with the container, and a second sparger connected or dimensioned to be connected to a source of a second gas composition different from the first gas composition in fluid communication with the container. The vessel further comprises a control system operatively associated with the first and second spargers and configured to operate the spargers independently of each other. Of course, third, fourth, fifth, or greater numbers of spargers can be included (e.g., greater than 10, or greater than 20 spargers), depending on, for example, the size of the container. In some embodiments, the vessel further comprises a mixing system including an impeller and a base plate, wherein the first and/or second sparger is associated with the base plate. In one particular embodiment, the first gas composition comprises air and the second gas composition comprises air supplemented with O₂ and N₂. If additional spargers are included, the spargers can be connected to a source of gas comprising N₂, CO₂, NH₃ and/or any other suitable gas.

Apertures associated with spargers can be formed in any suitable material. For instance, in one embodiment, a porous polymeric material is used as a sparging element to allow transport of gas from one side to another side of the material. Apertures can also be formed in other materials such as metals, ceramics, polymers, and/or combinations thereof. Materials having pores or apertures can have any suitable configuration. For example, the materials may be knitted, woven, or used to form meshes or other porous elements. The elements may be in the form of sheets, films, and blocks, for example, and may have any suitable dimension. In some cases, such elements are incorporated with impellers or impeller supports, e.g., as illustrated in FIG. 9. The elements can be positioned and held within regions of the impeller or impeller support securely enough for suitable use by any number of techniques including, for example, friction fitting, press fitting, detent mechanism, a clipping and clip release arrangement, fastening with screws, pegs, clamps, or the like, welding (e.g., heat and ultrasonic welding), and use of adhesives. In other embodiments, portions of the impeller and/or impeller support can be fabricated directly with pores or apertures that can allow fluids to flow therethrough.

The vessel may optionally include one or more sensors in electrical communication with the control system for determining an amount or concentration of a gas (e.g., O₂, N₂, CO₂, NH₃, a bi-product of a reaction) in the container. Additionally and/or alternatively, the vessel may include a sensor in electrical communication with the control system for determining a pH of a liquid in the container, or an amount or level of a foam in the bag.

In another aspect, a bubble column or airlift system (utilizing bubbles of air or other gas) may be used with the disposable bioreactor bag. Such a system may provide a mixing force by the addition of gas (e.g., air) near the bottom of the reactor. Here, the rising gas bubble and the lower density of gas-saturated liquid rise, displacing gas-poor liquid which falls, providing top-to-bottom circulation. The path of rising liquid can be guided, for example, using dividers inside the chamber of the bag. For instance, using a sheet of plastic which bisects the interior of the bioreactor bag, e.g., vertically, with a gap at the top and the bottom. Gas may be added on one side of the divider, causing the gas and gas-rich liquid to rise on one side, cross over the top of the barrier sheet, and descend on the other side, passing under the divider to return to the gas-addition point. In addition, such a bubble column/air-lift mixing system and method may be combined with any of the other mixing systems described herein.

In one aspect, a bioreactor system as described herein includes an enclosed resin loading/column packing system. Typically, column packing typically may be accomplished in a clean room with open carboys containing the resin which is manually mixed while the resin slurry is pumped onto the column. In one embodiment, however, a container such as a flexible container is loaded with chromatography resin which is slurried by an agitator while the slurry is pumped into a column.

In certain chemical, biochemical and/or biological processes requiring light, a bioreactor system described herein may include direct, indirect and/or piped-in lighting, e.g., using fiber-optics. Any suitable light source may be used. Such bioreactor systems may be useful for processing, for example, plant cells, e.g., to activate photosynthesis. In one particular embodiment, a phosphorescent flexible container is used to provide light, e.g., for growth of plant cells.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

What is claimed is:

1. A vessel, comprising:
a collapsible bag;
a reusable support structure supporting and containing the collapsible bag; and
a bladder or a compressible material positioned between an exterior wall of the collapsible bag and an interior wall of the support structure, the bladder or compressible material adapted to expand and/or contract so as to cause the collapsible bag to have a first configuration prior to expansion or contraction of the bladder or compressible material and a second configuration after expansion or contraction of the bladder or compressible material.

2. A vessel as in claim 1, comprising a bladder.

3. A vessel as in claim 1, comprising a compressible material.

4. A vessel as in claim 1, wherein the first configuration comprises the collapsible bag having a first volume and the second configuration comprises the collapsible bag having a second volume, the first volume being less than the second volume.

5. A vessel as in claim 1, wherein the first configuration comprises the collapsible bag having more wrinkles in a wall of the collapsible bag and the second configuration comprises the collapsible bag having less wrinkles in the wall of the collapsible bag.

6. A vessel as in claim 1, wherein the bladder or compressible material contains a gas.

7. A vessel as in claim 1, wherein the bladder contains a liquid.

8. A vessel as in claim 1, wherein the bladder or compressible material is not in fluid communication with any contents in the collapsible bag.

9. A vessel as in claim 1, wherein the bladder or compressible material is positioned around side walls of the collapsible bag.

10. A vessel as in claim 1, wherein the bladder or compressible material is positioned at a bottom portion of the collapsible bag.

11. A vessel as in claim 1, wherein a chemical, biological, or pharmaceutical reaction takes place within the vessel.

12. A vessel as in claim 1, further comprising a mixer.

13. A vessel, comprising:
a collapsible bag; and
a reusable support structure comprising at least one wall portion that can be expanded or compressed so as to cause the collapsible bag to have a first configuration prior to expansion or compression of the wall portion and a second configuration after expansion or compression of the wall portion.

14. (canceled)

15. A vessel as in claim 13, wherein the first configuration comprises the collapsible bag having more wrinkles in a wall of the collapsible bag and the second configuration comprises the collapsible bag having less wrinkles in the wall of the collapsible bag.

16-22. (canceled)

23. A method comprising:
positioning a collapsible bag in a reusable support structure such that the reusable support structure contains and supports the collapsible bag;
introducing a liquid into the collapsible bag; and
changing a pressure of a fluid in a region between an exterior wall of the collapsible bag and an interior wall of the support structure.

24. A method as in claim 23, wherein the changing step comprises creating a vacuum between the exterior wall of the collapsible bag and the interior wall of the support structure.

25-29. (canceled)

30. A vessel, comprising:
a collapsible bag;
a reusable support structure supporting and containing the collapsible bag; and
a detector adapted to determine the presence of leakage of a fluid from the collapsible bag.

31-33. (canceled)

34. A bioreactor system comprising:
a support structure;
a rigid container or a collapsible bag positioned within the support structure, the rigid container or collapsible bag including:
an impeller plate affixed to a lower portion of the rigid container or collapsible bag;
an impeller hub mounted on the impeller plate, the impeller hub having at least one impeller blade and having at least one magnet;
a motor having a shaft, the motor being provided adjacent to or within the support structure; and
a motor hub mounted on the motor shaft, the motor hub including at least one electromagnet, wherein upon mounting of the rigid container or collapsible bag within the support structure, the motor hub aligns with the impeller plate such that the electromagnet of the motor hub can drive the impeller hub when the motor shaft rotates; and
one or more sensors for sensing one or more parameters of any materials in the rigid container or collapsible bag.

35-41. (canceled)

42. A bioreactor system as in claim 34, wherein the impeller blade is positioned above the at least one porous element and extends across the width of the at least one porous element.

43. A bioreactor system as in claim 34, further comprising a thermally-conductive material embedded in at least a portion of a wall of the rigid container or collapsible bag.
